(19) United States
(12) Patent Application Publication
(10) Pub. No.: US 2020/0237909 A1
(43) Pub. Date: Jul. 30, 2020

(54) METHODS OF TREATING OR PREVENTING BREAST CANCER METASTASES USING ANTIBODIES TO JAGGED1 AND CHEMOTHERAPEUTIC AGENTS

(71) Applicant: The Trustees of Princeton University, Princeton, NJ (US)

(72) Inventors: Yibin KANG, Princeton, NJ (US); Hanqiu ZHENG, Franklin Park, NJ (US)

(73) Assignee: The Trustees of Princeton University, Princeton, NJ (US)

(21) Appl. No.: 16/755,250

(22) PCT Filed: Oct. 11, 2018
(86) PCT No.: PCT/US18/55387

§ 371 Date: Apr. 10, 2020

Related U.S. Application Data
(60) Provisional application No. 62/571,653, filed on Oct. 12, 2017.

(51) Int. Cl. A61K 39/395; A61P 35/04; A61K 45/06; A61K 31/337; A61K 31/513; A61K 33/243

(52) U.S. Cl. CPC A61K 39/39558; A61P 35/04; A61K 45/06; A61K 2039/505; A61K 31/513; A61K 33/243; A61K 31/337

(57) ABSTRACT

Provided is a method of combination therapy for prevention or treatment of cancer bone metastasis comprising administering to a patient in need thereof a therapeutically effective amount of an anti-Jagged1 antibody or an antigen-binding fragment thereof and a chemotherapeutic agent. The combination therapy achieves an excellent synergistic effect. In preferred embodiments, the patient is a human female and the cancer is breast cancer.

### CROSS REFERENCE TO RELATED APPLICATIONS

This application claims priority from U.S. Provisional Patent Application Ser. No. 62/571,653, filed Oct. 12, 2017, the content of which is hereby incorporated by reference, in its entirety.

### STATEMENT REGARDING FEDERALLY SPONSORED RESEARCH OR DEVELOPMENT

This invention was made with government support under Grant No. CA212410 awarded by the National Institutes of Health. The government has certain rights in the invention.

### FIELD

The present disclosure concerns prevention or treatment of cancer bone metastases with a combination of an anti-Jagged1 antibody or antigen-binding fragment thereof and chemotherapy. In particular, the disclosure concerns the treatment of breast cancer that has metastasized to bone or has the potential to metastasized to bone.

### BACKGROUND

Breast cancer is the most common human female malignancy and the second leading cause of cancer-related death in the United States. Among late stage breast cancer patients, more than 70% suffer from bone metastasis, which is often accompanied by severe bone pain, fracture and potentially lethal complications such as hypercalcemia (Weilbaecher et al., 2011). Although radiotherapy, chemotherapy and anti-osteolytic agents such as bisphosphonate and RANKL antibody denosumab can reduce morbidity associated with bone metastasis, these treatments often do not significantly extend the survival time of the patients or provide a cure (Coleman et al., 2008), as metastatic cancers often acquire resistance to these treatments.

Tumor-stromal interaction plays a major role in promoting bone metastasis of breast cancer (Weilbaecher et al., 2011). The bone microenvironment contains a great variety of stromal cell types, such as osteoblasts, osteoclasts, mesenchymal stem cells (MSCs), and hematopoietic cells. While previous research has focused on the cross-communication between breast cancer cells and bone resorbing osteoclasts, the contributions of other stromal cell types to bone metastasis are less studied. Among the supporting stromal cells, bone-building osteoblasts have recently been shown to constitute an osteogenic niche that is critical for the survival and colonization of disseminated tumor cells in the bone (Shiozawa et al., 2011; Wang et al., 2015). Despite these recent progresses, our molecular understanding of the interaction between tumor cells and osteoblastic cells in the bone niche remain largely incomplete. For example, how such tumor-niche interactions contribute to the resistance of metastatic breast cancer to standard bone metastasis treatments, such as chemotherapy, remains poorly understood.

In human breast cancer, elevated expression of Jagged1 and Notch1, but not other Notch pathway ligands or receptors, is significantly associated with poor prognosis (Reedijk et al., 2005; Reedijk et al., 2008; Sethi et al., 2011). Functionally, studies have started to appreciate Jagged1 as a critical mediator of tumor-stromal interactions. For example, Jagged1 activates Notch signaling in endothelial cells to promote angiogenesis in head and neck cancer (Zeng et al., 2005). Our previous study identified tumor-derived Jagged1 as a bone metastasis-promoting factor by activating Notch signaling in osteoblasts to increase the production of Interleukin-6 (IL-6) and connective tissue growth factor (CTGF), which feeds back to tumor cells to promote proliferation and survival. Meanwhile, Jagged1 stimulates osteoclastogenesis and bone degradation, leading to the release of bone-derived growth factors including TGF-β, a potent inducer of Jagged1 expression in tumor cells, thus forming a positive feedback cycle (Sethi et al., 2011).

Several therapeutic strategies have been developed to target the Notch signaling pathway. Most of the inhibitors were designed to target γ-secretase, which mediates the proteolytic cleavage of Notch receptors to generate signal-transducing Notch intracellular domain (NICD), a necessary step in Notch pathway activation upon ligand binding (Rizzo et al., 2008). However, γ-secretase inhibitors (GSIs) have been reported to induce severe gastrointestinal (GI) track toxicity (Imbimbo, 2008), preventing further clinical development of this class of inhibitors. Recent studies indicated that targeting individual Notch receptors or ligands can potentially achieve therapeutic effect without causing severe GI tract toxicity (Choy et al., 2017; Ridgway et al., 2006; Wu et al., 2010).

### SUMMARY

Disclosed herein are methods of preventing or treating cancer bone metastases by administering to a patient in need thereof a therapeutically effective amount of an anti-Jagged1 antibody or antigen-binding fragment thereof and a chemotherapeutic agent.

In some embodiments, administration of the anti-Jagged1 antibody or antigen-binding fragment thereof and the chemotherapeutic agent results in a synergistic effect.

In some embodiments, the cancer is breast cancer, prostate cancer, lung cancer, ovarian cancer, colorectal cancer, melanoma, multiple myeloma, thyroid cancer, bladder cancer, or kidney cancer.

In some embodiments, the patient has been diagnosed with breast cancer that has metastasized to bone. In some embodiments, the patient has been diagnosed with breast cancer but the breast cancer has not metastasized to bone.

In some embodiments, the patient is a mammal. In some embodiments, the patient is a human. In some embodiments, the patient is a female human. In some embodiments, the patient is a female human and the Jagged1 is human Jagged1.

In some embodiments, the anti-Jagged1 antibody is a monoclonal antibody. In some embodiments, the monoclonal antibody is a fully human monoclonal antibody, a humanized monoclonal antibody, or a chimeric monoclonal antibody.

In some embodiments, the chemotherapeutic agent is selected from the group consisting of alkylating agents, alkyl sulfonates, aziridines, ethylenimines, methylamelamines, colchicines, camptothecins, nitrogen mustards, nitrosoureas, plant alkaloids, bisphosphonates, anthracyclines, anti-metabolites, anti-microtubule agents, topoisomerase inhibitors, cytotoxic antibiotics, metal salts, toxoids, taxanes, pyrimidine analogs, purine analogs, aromatase inhibitors, mitomycins, androgens, anti-adrenals, folic acid replenishers, anti-folates, dihydrofolate reductase inhibitors, thymidylate synthase inhibitors, vinca alkaloids, and anti-hormonal agents, as well as pharmaceutically acceptable salts, acids, or derivatives of any of the above, as well as combinations of two or more of the above.

In some embodiments, the chemotherapeutic agent is TAXOL® (paclitaxel), docetaxel, ADRIAMYCIN® (doxorubicin), epirubicin, 5-fluorouracil, CYTOXAN® (cyclophosphamide), carboplatin, PLATINOL® (cisplatin), IBRANCE® (palbociclib), ARIMIDEX® (anastrozole), AVASTIN® (bevacizumab), XELODA® (capecitabine), DOXIL® (doxorubicin liposomal injection), AROMASIN® (exemestane), GEMZAR® (gemcitabine), IXEMPRA® (ixabepilone), FEMARA® (letrozole), or HERCEPTIN® (trastuzumab).

In some embodiments, progression free survival of the patient is extended beyond that provided by administering the anti-Jagged1 antibody or antigen-binding fragment thereof alone or the chemotherapeutic agent alone. In some embodiments, the progression free survival is extended in a synergistic manner as compared to the progression free survival provided by administering the anti-Jagged1 antibody or antigen-binding fragment thereof alone or the chemotherapeutic agent alone.

In some embodiments, overall survival of the patient is extended beyond that provided by administering the anti-Jagged1 antibody or antigen-binding fragment thereof alone or the chemotherapeutic agent alone. In some embodiments, the overall survival is extended in a synergistic manner as compared to the overall survival provided by administering the anti-Jagged1 antibody or antigen-binding fragment thereof alone or the chemotherapeutic agent alone.

In some embodiments, the methods disclosed herein extend the progression free survival and the overall survival of patients treated with the combinations of anti-Jagged1 antibody or antigen-binding fragment thereof and chemotherapeutic agent disclosed herein. In some embodiments, the progression free survival and the overall survival are extended in a synergistic manner as compared to the progression free survival and the overall survival provided by administering the anti-Jagged1 antibody or antigen-binding fragment thereof alone or the chemotherapeutic agent alone.

In some embodiments, the administration of the anti-Jagged1 antibody or antigen-binding fragment thereof and the chemotherapeutic agent does not lead to an abnormal increase of trabecular bone density in the patient.

Disclosed herein is a method of providing a synergistic effect in the treatment of breast cancer comprising administering to a human female having breast cancer a therapeutically effective amount of an anti-Jagged1 antibody or antigen-binding fragment thereof and a chemotherapeutic agent, wherein the progression free survival and/or the overall survival of the human female is extended beyond that provided by administering the anti-Jagged1 antibody or antigen-binding fragment thereof alone or the chemotherapeutic agent alone.

In some embodiments, the anti-Jagged1 antibody or antigen-binding fragment thereof and the chemotherapeutic agent are administered concurrently. In some embodiments, the anti-Jagged1 antibody or antigen-binding fragment thereof is administered before the chemotherapeutic agent. In some embodiments, the anti-Jagged1 antibody or antigen-binding fragment thereof is administered after the chemotherapeutic agent.

In some embodiments, the anti-Jagged1 antibody or antigen-binding fragment thereof and the chemotherapeutic agent are administered in the same pharmaceutical formulation. In some embodiments, the anti-Jagged1 antibody or antigen-binding fragment thereof and the chemotherapeutic agent are administered in separate pharmaceutical formulations.

### BRIEF DESCRIPTION OF THE FIGURES

FIG. 1. Characterization of fully human monoclonal antibodies against Jagged1. (A) Kinetic exclusion assay for binding of 15D11 with murine Jagged1 protein. Increasing number (1.5×10² to 9×10⁶) of 293T cells with murine Jagged1 expression (Clone #1) were added into the solution with fixed amount of 15D11 antibody (10 or 100 pM) and remaining amount of antibody in the liquid was determined after 4 hours. (B) A Notch reporter luciferase assay was used to determine IC50 for 15D11 in Jagged1-induced Notch activation. Human TE671 expressing a multimerized 7XCSL-responsive element upstream of a minimal promoter (Notch reporter) were co-cultured with HEK293 expressing Jagged1. 15D11 (0.07-1333.4 nM) was added to the cells for 2 days. Luciferase signal was quantified. The percent inhibition was calculated relative to signals of reactions lacking 15D11 and 293T co-culture. (C) RAW264.7 pre-osteoclast cells were seeded on Fc- or recombinant Jagged1 coated plates in the presence of 5 ng/ml RANKL and indicated concentrations of IgG or 15D11. Cells were cultured for 5-7 days before fixing for TRAP staining to visualize multi-nucleated mature osteoclasts. (D) Quantification of TRAP-positive osteoclasts per field from C. Data is presented as mean±SEM. n=3. **p<0.01 by Student's t-test. (E) SCP28-Vector or SCP28-Jagged1 cells were co-cultured with RAW264.7 cells in the presence of 5 ng/ml RANKL and 1 μg/ml IgG or 15D11. Cells were cultured for 5-7 days before fixing for TRAP staining to visualize multi-nucleated mature osteoclasts. (F) Primary bone marrow cells were flushed from tibias of 4-6 week old wild-type FVB mice and plated for 24 h, after which the non-adherent cells were collected and cultured in M-CSF (50 ng/mL) for 2 days and then co-cultured with SCP28-Vector or SCP28-Jagged1 in the presence of RANKL (20 ng/ml) and 1 μg/ml IgG or 15D11. Cells were allowed to differentiate into osteoclasts for additional 4-5 days before fixing and TRAP staining. (G) mRNA expression of mouse IL-6 and Hes1 in MC3T3 cells co-cultured with SCP28-vector control or SCP28-Jagged1 cells, treated with either IgG control or 15D11. Primers used in this experiment were specifically designed to only detect mouse genes, but not human genes. n=3. **p<0.01 by Student's t-test. (H) Left panel: Expression of IL-6 protein in cell lysate and conditional media taken from MC3T3 cells co-cultured with SCP28-vector control or SCP28-Jagged1 overexpressing cells. Recombinant IL-6 protein served as positive control. Right Panel: IL-6 protein expression in conditional media from MC3T3 cells co-cultured with SCP28-vector control or SCP28-Jagged1 cells, treated with either IgG control or 15D11 antibody. β-actin served as loading control. Bars below the IL-6 immunoblots represent relative band density of IL-6 as quantified by ImageJ software. Size bars in C, E, and F: 100 μm.

FIG. 2. 15D11 inhibits tumor-derived Jagged1 dependent bone metastasis. (A) Schematic representation of the experiment. (B) Representative BLI images on Day 1 immediately after the IC injection and on Day 35 at the end point of the experiment. (C) Quantification of bone metastasis burden each week based on BLI imaging from the experiment. n=10. *p<0.05 by Mann-Whitney test. (D) Representative X-ray images and Quantification of osteolytic lesions. AU: arbitrary unit. n=8. **p<0.01 by Student's t-test. (E) Representative X-ray, μCT, H&E staining and TRAP staining images from the experiment. Size bars: 100 μm for H&E and 25 μm for TRAP.

FIG. 3. Synergistic inhibition of bone metastasis by SCP28-Jagged1 with combined treatment of 15D11 and chemotherapy. (A) Schematic representation of the experiment. (B) Quantification of bone metastasis burden each week based on BLI imaging. n=10. *p<0.05 and **p<0.01 by Mann-Whitney test. (C) Representative BLI images and X-ray images at week 7. (D) Representative H&E and TRAP staining images from the experiment. (E) Quantification of osteolytic lesions based on X-ray images. n=10. **p<0.01 by Student's t-test. (F) Quantification of TRAP+ osteoclasts from decalcified histological bone sections of hind limbs from mice, n=10. **p<0.01 by Student's t-test. Size bar: 100 μm for H&E and 50 μm for TRAP.

FIG. 4. Synergistic inhibition of bone metastasis by SCP28 with combined treatment of 15D11 and chemotherapy. (A) Schematic illustration of the experimental procedure. (B) Quantification of bone metastasis burden based on BLI imaging. n=10. *p<0.05 and **p<0.01 by Mann-Whitney test. (C) Representative BLI images and X-ray images at week 6. (D) Representative H&E and TRAP staining images from the experiment. (E) Quantification of osteolytic lesions based on X-ray images. n=10. **p<0.01 by Student's t-test. (F) Quantification of TRAP+ osteoclasts from decalcified histological bone sections of hind limbs from mice, n=10. **p<0.01 by Student's t-test. Size bar: 100 μm for H&E and 50 μm for TRAP.

FIG. 5. Chemotherapy induces Jagged1 expression in osteoblast lineage cells. (A) Jagged1 mRNA expression in osteoblast MC3T3-E1-Clone #4 osteoblast cells and mesenchymal stem cells (MSC), HPMECL endothelial cells (endo), RAW264.7 pre-osteoclast cells, and SCP28 and SUM1315-M1B1 breast tumor cells 48 hr post-treatment with 25 nM paclitaxel or 10 μM cisplatin. (B) Athymic nude mice were i.v. injected with PBS or cisplatin and euthanized 48 hr later. Hind limb bones were collected for IF staining against Alp (green) and Jagged1 (red). Nuclei were counter-stained with DAPI (blue). (C-E) Athymic nude mice were treated with either PBS or cisplatin on Day 0. One day after the pre-treatment, MCF7 (C), SUM1315-M1B1 (D) or SCP28 (E) cells were delivered locally to the hind limbs of these mice by IIA injection. BLI imaging and quantification of BLI signal at Day 4. (F) Mice from experiment in E were euthanized after BLI imaging at Day 4. Hind limbs were fixed and processed for IF imaging of GFP-expressing tumor cells (green) and ALP positive osteoblasts (red). Size bar: 80 μm. In A: n=3. *p<0.05 by Student's t-test. In C-E: n=5. *p<0.05 by Student's t-test. #: statistically insignificant.

FIG. 6. Osteoblast-derived Jagged1 promotes bone metastasis seeding and progression. (A) Representative μCT imaging and H&E staining images from FVB WT and Col1a1-Jagged1 mice. Yellow box: trabecular bone areas of Col1a1-Jagged1 mice showed decreased bone density (in μCT imaging) and were filled with bone marrow cells (in H&E staining). Size bar: 0.5 mm in μCT and 200 μm in H&E. (B) Col1a1-Jagged1 mice were treated with IgG or 15D11 twice a week for 3 months. Mice were then euthanized for bone sample collection and histology analysis. Representative TRAP staining images from IgG or 15D11 group were presented. Size bar: 200 μm. (C) PyMT-A-FIG cells were delivered locally to the hind limbs of WT and Col1a1-Jagged1 mice by intra-tibial (i.t.) injection. Three weeks after injection, the engraftment of breast cancer cells in the bone was assessed by BLI. n=4. **p<0.01 by Student's t-test. (D) PyMT-A-FIG cells were delivered locally to the hind limbs of WT and Col1a1-Jagged1 mice by IIA injection. Tumor cells in the bone were quantified based on BLI imaging at Day 1 and Day 4 (right panel). n=5. *p<0.05 by Student's t-test. (E) IF staining of bone samples at Day 4 from experiment in C, showing GFP-expressing tumor cells (green) and ALP-positive osteoblasts (red). Size bar: 80 μm.

FIG. 7. Pro-survival effect of osteoblast Jagged1 on cancer cells can be blocked by 15D11. (A) 3-D tumorsphere culture of MCF7, SCP28, and SUM1315-M1B1 cells, alone or in co-cultured with osteoblast cells, were treated with indicated concentrations of cisplatin or docetaxel. Two days after the chemotherapy, the surviving tumor spheres were counted under the fluorescent microscope. n=3. *p<0.05 by Student's t-test. (B) Cell culture of SCP28 cells alone or in co-culture with MC3T3-E1 pre-osteoblast cells were treated with PBS control or cisplatin. Protein samples were collected at 6 hr, 24 hr, and 72 hr later. Cleaved PARP and cleaved caspase-3 were determined by immunoblotting. # marks full length PARP and * marks cleaved PARP. (C) Real-time PCR analysis of Notch downstream gene (human specific HEY1, HEY2, and HES2) in tumor cells co-cultured with MSC and treated with respective chemotherapeutic agents. n=3. *p<0.05 by Student's t-test. (D) Microarray analysis was performed with mRNA from cell culture of SUM1315-M1B1 cells alone or in co-culture with MC3T3-E1 osteoblasts, with or without cisplatin treatment. Heatmap shows 16 apoptosis-related genes with at least 2-fold changes in the cisplatin treatment condition (tumor cell culture alone vs. co-culture). Five p53 pathway-related genes were highlighted in red. (E-F) Similar experiment as in FIG. 4A using SCP28 were performed, except that the mice were euthanized after two treatments. Hind limb bones were fixed and cryo-sectioned for IF staining against CC3 for apoptotic cells and GFP for tumor cells (E), and CC3 positive apoptotic cells among GFP-positive tumor cells were quantified in F. Size bar: 25 μm. n=5. **p<0.01 by Student's t-test. (G) Athymic nude mice were treated with either PBS or cisplatin via i.v. injection on Day 0. On Day 1, PBS pre-treated mice were treated with IgG, while cisplatin pre-treated mice were treated with either IgG or 15D11. SUM1315-M1B1 cells were delivered locally to the hind limbs of these nude mice by IIA injection. Tumor cells in the bone were quantified based on BLI imaging at Day 1 and Day 4. BLI signal representing initial bone metastatic seeding at Day 4 was normalized to BLI signal intensity at Day 1 (lower panel). n=5. *p<0.05 by Student's t-test. (H) Mice from experiment in E were euthanized after BLI imaging at Day 4. Hind limbs were fixed and processed for IF imaging of GFP-expressing tumor cells (green) and ALP-positive osteoblasts (red). Size bars: 40 μm.

FIG. 8. Combination of 15D11 and chemotherapy strongly inhibits spontaneous bone metastasis. (A) Paired bone marrow cytospin samples were collected from cancer patients before and after cisplatin+paclitaxel chemotherapy. Slides were IF co-stained for ALP-positive osteoblasts (red) and Jagged1 (green). Size bar: 40 μm. (B) Normalized signal intensity of Jagged1 staining to ALP staining. Signal intensity was quantified using ImageJ software (NIH). n=14. *p<0.05 by Student's t-test. (C) Schematic representation of experimental procedures. 4T1.2 mouse mammary tumor cells were injected in the mammary fat pad of 6-8 week old female Balb/c mice. Two weeks later when the primary tumors progressed to about 5-8 mm in diameter, mice were treated with IgG, 15D11, paclitaxel, or both 15D11 and paclitaxel twice a week. Spontaneous bone metastasis progression was monitored by weekly X-ray images and by histological H&E staining and TRAP staining. (D) X-ray images of these experimental mice at Week 5. (E) Quantification of the percentage of mice developed spontaneous bone metastasis based on X-ray and H&E staining at the end point. (F) Quantification of osteolytic lesions based on X-ray images. AU: arbitrary unit. n=8. **p<0.05, **p<0.01 by Student's t-test. (G) Schematic model for Jagged1-Notch signaling in bone metastasis progression and chemoresistance. Tumor-derived Jagged1 promote bone metastasis by engaging osteoblasts and osteoclasts (upper panel), while chemotherapy-induced Jagged1 in osteogenic cells promotes tumor cell survival under chemotherapy (lower panel). 15D11 targets both processes and synergizes with chemotherapy to strongly reduce bone metastasis.

FIG. 9. 15D11 synergizes with chemotherapy to inhibit bone metastasis in the SUM1315-M1B1 model. (A) Western blot analysis of Jagged1 protein expression level in SUM1315-M1B1, SPC2, SCP28-Vector, and SCP28-Jagged1 cells. β-actin was used as loading control. (B) Schematic representation of the experimental procedure. In brief, SUM1315-M1B1 cells were IC injected into 4-6 weeks female athymic nude mice to generate bone metastases. One week later, mice were randomized into four groups and treated with respective therapeutic agents. Bone metastasis progression was monitored by weekly BLI imaging until the experimental endpoint (4 weeks after injection). (C) Representative BLI and X-ray images of mice in Week 4 right before euthanizing the mice. (D) Quantification of bone metastasis burden each week based on BLI imaging from the experiment. Notice there was a minor reduction of BLI signal in either 15D11 treatment group or in paclitaxel treatment group, which did not reach the statistical significance. Combination of 15D11 and OPG treatment significantly reduced bone metastasis burden at Week 4. n=10, p<0.01 by Mann-Whitney test. (E) Representative H&E staining and TRAP staining images of hind limb bones from experiment in A were shown. Scale bar: 100 μm. (F) Quantification of osteolytic lesions based on X-ray images. AU: arbitrary unit. n=10, **p<0.01 by Student's t-test. (G) Quantification of the number of TRAP-positive osteoclasts per field. n=5, **p<0.01 by Student's t-test.

FIG. 10. Administration of 15D11 sensitizes bone metastatic tumor cells to chemotherapy in vitro and in vivo. (A) A 3-D co-culture model for breast cancer cells and osteoblasts or MSCs in low attachment plates. At the presence of osteoblasts or MSCs (red), tumor cells (green) formed hybrid tumorspheres in close contact with osteoblast/MSCs. Scale bar: 100 μm (B) MSCs were cultured in regular media on 10 cm dishes and treated with 10 μM cisplatin for 24 hr. Cells were then switched to serum free media for another 24 hr before the CM were collected. Luciferase-labeled SUM1315-M1B1 cells were seeded alone, with MSC CM, or with MSCs and treated with 10 μM cisplatin. The relative number of surviving tumor cells after 48 hr were quantified by luciferase assay. n=3, *p<0.05 by Student's t-test. (C-D) 3-D tumorsphere culture of SUM1315-M1B1 cells, alone or in co-cultured with STA endothelial cells, were treated with indicated concentrations of cisplatin or docetaxel. Two days after the chemotherapy, the surviving tumor spheres were counted under the fluorescent microscope. n=3, n.s., p>0.05 by Student's t-test. (E) In SCP28 and MSC 3-D co-culture model, established tumorspheres were treated with respective chemotherapy agent in combination with 5 μg/ml IgG or 15D11. The surviving tumorspheres were counted under the fluorescence microscope (for GFP positive tumorspheres). n=3, *p<0.05, *p<0.05 by Student's t-test. (F) In SUM1315-M1B1 and MSC 3-D co-culture model, established tumorspheres were treated with respective chemotherapy agent in combination with 5 μg/ml IgG or 15D11. The surviving GFP positive tumorspheres were counted under the fluorescence microscope. n=3, *p<0.05 by Student's t-test. (G) Female athymic nude mice were IP injected with PBS or Cisplatin one day before tumor cell injection. 10⁵ SUM1315-M1B1 cells were IIA injected into these nude mice. Cisplatin treated mice were randomly assigned into two groups and treated with either IgG or 15D11 antibody at the time of tumor cell injection. The representative BLI images from Day 4 were shown. (H) Quantification of BLI signal at Day 4 from experiment in E. n=5, *p<0.05, **p<0.01 by Student's t-test.

### DETAILED DESCRIPTION

“Administering” refers to delivering a therapeutically effective substance such as the anti-Jagged1 antibodies or antigen-binding fragments thereof disclosed herein and/or a chemotherapeutic agent to a patient by any method known in the art that may achieve the result sought. “Administering” may be, e.g., subcutaneous, intravenous, intramuscular, intralesional, intraperitoneal, liposome-mediated, transmucosal, intestinal, topical, nasal, oral, anal, ocular or otic.

“Anti-Jagged1 antibody or antigen-binding fragment thereof” refers to an antibody or antigen-binding fragment thereof that is capable of binding Jagged1 with sufficient affinity that the antibody or antigen-binding fragment thereof is useful as a therapeutic agent for the treatment of cancer bone metastasis, in particular for the treatment of metastases from breast cancer. In some embodiments, the “anti-Jagged1 antibody or antigen-binding fragment thereof” prevents Jagged1 from binding to a Notch receptor and/or activating a Notch signaling pathway.

“Chemotherapeutic agent” refers to a substance, preferably a chemical compound having a molecular weight<1,000 daltons, useful in the treatment of cancer.

“Concurrently” refers to administering an anti-Jagged1 antibody or an antigen-binding fragment thereof and a chemotherapeutic agent to a patient at the same or approximately the same time, e.g., within a few minutes of one another. The antigen-binding fragment thereof and a chemotherapeutic agent may be administered by the same or by different routes (e.g., each may be administered intravenously or one may be administered intravenously and one may be administered subcutaneously).

“Patient” refers to a mammal, preferably a human, particularly a female human, but can also be companion animals such as dogs or cats, or farm animals such as horses, cattle, pigs, or sheep.

“Synergistic effect” refers to a greater than additive therapeutic benefit to a patient when an anti-Jagged1 antibody or antigen-binding fragment thereof and a chemotherapeutic agent are administered to the patient, as compared to the sum of the therapeutic benefits of the anti-Jagged1 antibody or antigen-binding fragment thereof and the chemotherapeutic agent when each is administered to the patient in the absence of the other.

“Therapeutically effective amount” refers to an amount of an anti-Jagged1 antibody or antigen-binding fragment thereof and chemotherapeutic agent effective to treat cancer bone metastasis in the patient. The therapeutically effective amount may, inter alia, reduce the number of cancer cells that metastasize to bone, reduce the size of metastases, inhibit (i.e., slow to some extent and preferably stop) growth of cancer cells that have metastasized to bone, and/or relieve to some extent one or more of the symptoms associated cancer bone metastases.

Previous studies of chemoresistance mostly focused on tumor-intrinsic mechanisms, such as reduced drug uptake, increased drug efflux, alterations in drug-target interaction, changes in cellular response, in particular increased cell ability to repair DNA damage or tolerate stress conditions, and defects in apoptotic pathways. The tumor microenvironment, especially the unique stromal niches in the organ sites of metastasis, has received comparatively little attention.

The present disclosure is based in part on the inventors' discovery that the microenvironment of cancer bone metastases, in particular the expression of Jagged1 in osteogenic cells (osteoblasts and/or mesenchymal stem cells (MSCs)), plays a key role in the establishment, survival, and proliferation of cancer cells that have metastasized to bone and, in particular, that have been subject to chemotherapy.

The present disclosure also is based in part on the inventors' discovery that chemotherapeutic agents stimulate the expression of Jagged1 on osteoblasts and MSCs during treatment of cancer bone metastases, leading to the eventual lack of effectiveness of those agents. By combining chemotherapy with an anti-Jagged1 antibody or antigen-binding fragment thereof, the inventors discovered that a synergistic effect may be obtained, wherein the effectiveness of the two therapeutic agents far outstrips the sum of the effectiveness of each agent when administered alone, without the other.

This synergistic effect of administering both an anti-Jagged1 antibody or antigen-binding fragment thereof and a chemotherapeutic agent allows for the use of a lower amount of chemotherapeutic agent than is generally used, leading to fewer of the debilitating side effects typically associated with chemotherapy.

In some embodiments, the anti-Jagged1 antibody used in the methods disclosed herein is humanized, chimeric, or fully human (where the fully human antibody is not found in nature). In some embodiments, the antigen-binding fragment thereof may be, e.g., Fab, F(ab′)2, diabody, FV, scFv.

In some embodiments, the anti-Jagged1 antibody or antigen-binding fragment thereof used in the methods disclosed herein is an antibody or antigen-binding fragment thereof disclosed in U.S. Pat. No. 8,802,103 (the contents of which are incorporated by reference herein for that purpose), including 64M14.

In other embodiments, the anti-Jagged1 antibody or antigen-binding fragment thereof used in the methods disclosed herein is an antibody or antigen-binding fragment thereof disclosed in U.S. Pat. No. 9,416,178 (the contents of which are incorporated by reference herein for that purpose), including 64M51, 64R7, 64R1B, 133R0201, 133R0203, and 133R0205.

In some embodiments, the anti-Jagged1 antibody or antigen-binding fragment thereof from U.S. Pat. No. 9,416,178 used in the methods disclosed herein comprises a heavy chain comprising the following CDRs:

a CDR1 comprising
(SEQ ID NO: 1)
SYAMH;
a CDR2 comprising a sequence selected from
the group consisting of
(SEQ ID NO: 2)
VISYDGSNKYYADSVKG,
(SEQ ID NO: 3)
AIYPDSSNKYYADSVKG,
(SEQ ID NO: 4)
AISPEASNKYYADSVKG,
and
(SEQ ID NO: 5)
AIYPASSNKYYADSVKG;
and
a CDR3 comprising
(SEQ ID NO: 6)
DKYDIPDAFDI.

The heavy chain CDRs immediately above may be combined with a light chain comprising the following light chain CDRs:

(SEQ ID NO: 7)
a CDR1 comprising RASQGISNDLA;
(SEQ ID NO: 8)
a CDR2 comprising ATSTLQS;
and
(SEQ ID NO: 9)
a CDR3 comprising QQSYNAPI.

In some embodiments, the anti-Jagged1 antibody or antigen-binding fragment thereof from U.S. Pat. No. 9,416,178 used in the methods disclosed herein comprises a heavy chain comprising the following CDRs:

(SEQ ID NO: 10)
a CDR1 comprising SSNWWS;
(SEQ ID NO: 11)
a CDR2 comprising EIFHGENTNYNPSLKS;
and
(SEQ ID NO: 12)
a CDR3 comprising NPGIGAAKFDS.

The heavy chain CDRs immediately above may be combined with a light chain comprising the following light chain CDRs:

(SEQ ID NO: 13)
a CDR1 comprising KSSQSLLHSDGKTYLY;
(SEQ ID NO: 14)
a CDR2 comprising EVSNRFS;
and
(SEQ ID NO: 15)
a CDR3 comprising MQHIDFP.

In some embodiments, the anti-Jagged1 antibody or antigen-binding fragment thereof from U.S. Pat. No. 9,416,178 used in the methods disclosed herein comprises a heavy chain comprising the following CDRs:

(SEQ ID NO: 16)
a CDR1 comprising SYWIH;
(SEQ ID NO: 17)
a CDR2 comprising RIYPGIGSTYYNEKFKD;
and
(SEQ ID NO: 18)
a CDR3 comprising NGGFFDY.

The heavy chain CDRs immediately above may be combined with a light chain comprising the following light chain CDRs:

a CDR1 comprising
(SEQ ID NO: 19)
RASESVDSYGNSFMH;
a CDR2 comprising
(SEQ ID NO: 20)
RASNLES;
and
a CDR3 comprising
(SEQ ID NO: 21)
QQSNEDPWT.

In other embodiments, the anti-Jagged1 antibody or antigen-binding fragment thereof used in the methods disclosed herein is an antibody or antigen-binding fragment thereof disclosed in U.S. Patent Application Publication No. 2015/0232568 (the contents of which are incorporated by reference herein for that purpose).

In some embodiments, the anti-Jagged1 antibody or antigen-binding fragment thereof from U.S. Patent Application Publication No. 2015/0232568 used in the methods disclosed herein comprises a heavy chain comprising the following CDRs:

a CDR1 comprising
(SEQ ID NO: 22)
GFTFSNYGIH;
a CDR2 comprising
(SEQ ID NO: 23)
WITPDGGYTDYADSVK;
and
a CDR3 comprising
(SEQ ID NO: 24)
AGSWFAY.

The heavy chain CDRs immediately above may be combined with a light chain comprising the following light chain CDRs:

a CDR1 comprising
(SEQ ID NO: 25)
RASQDVSTAVA;
a CDR2 comprising
(SEQ ID NO: 26)
SASFLYS;
and
a CDR3 comprising
(SEQ ID NO: 27)
QQSYTTPPT.

In some embodiments, the anti-Jagged1 antibody or antigen-binding fragment thereof from U.S. Patent Application Publication No. 2015/0232568 used in the methods disclosed herein comprises a heavy chain comprising the following CDRs:

a CDR1 comprising
(SEQ ID NO: 28)
GFTFTSYDIH;
a CDR2 comprising
(SEQ ID NO: 29)
GISPADGDTDYANSV;
and
a CDR3 comprising
(SEQ ID NO: 30)
NDYDVRSVGSGMDY.

The heavy chain CDRs immediately above may be combined with a light chain comprising the following light chain CDRs:

a CDR1 comprising
(SEQ ID NO: 31)
RASDVSTAVA;
a CDR2 comprising
(SEQ ID NO: 32)
SASFLYS;
and
a CDR3 comprising
(SEQ ID NO: 33)
QQSYTTPPT.

In other embodiments, the anti-Jagged1 antibody or antigen-binding fragment thereof used in the methods disclosed herein is an antibody or antigen-binding fragment thereof disclosed in U.S. Pat. No. 9,725,518 (the contents of which are incorporated by reference herein for that purpose), including J1-65D, J1-156A, J1-183D, and J1-187B.

In some embodiments, the anti-Jagged1 antibody or antigen-binding fragment thereof from U.S. Pat. No. 9,725,518 used in the methods disclosed herein comprises a heavy chain comprising the following CDRs:

a CDR1 comprising
(SEQ ID NO: 34)
DYEMH;
a CDR2 comprising
(SEQ ID NO: 35)
QPGGGGTAYNQKFKG;
and
a CDR3 comprising
(SEQ ID NO: 36)
RGYDDYPFAY.

The heavy chain CDRs immediately above may be combined with a light chain comprising the following light chain CDRs:

a CDR1 comprising
(SEQ ID NO: 37)
RASGNIHNYLA;
a CDR2 comprising
(SEQ ID NO: 38)
NAKTLADDI;
and
a CDR3 comprising
(SEQ ID NO: 39)
NHFWSAPWT.

In some embodiments, the anti-Jagged1 antibody or antigen-binding fragment thereof from U.S. Pat. No. 9,725,518 used in the methods disclosed herein comprises a heavy chain comprising the following CDRs:

a CDR1 comprising
(SEQ ID NO: 40)
DYAMH;
a CDR2 comprising
(SEQ ID NO: 41)
DTYYGDASYNQKFKG;
and
a CDR3 comprising
(SEQ ID NO: 42)
LYDYDGGFAY.

The heavy chain CDRs immediately above may be combined with a light chain comprising the following light chain CDRs:

a CDR1 comprising
(SEQ ID NO: 43)
KSSQSLLNSSNQKNYL;
a CDR2 comprising
(SEQ ID NO: 44)
FASTRRSGV;
and
a CDR3 comprising
(SEQ ID NO: 45)
QQHYSTPYT.

In some embodiments, the anti-Jagged1 antibody or antigen-binding fragment thereof from U.S. Pat. No. 9,725,518 used in the methods disclosed herein comprises a heavy chain comprising the following CDRs:

a CDR1 comprising
(SEQ ID NO: 46)
DYAIH;
a CDR2 comprising
(SEQ ID NO: 47)
NTYYGDSKYNQKFKD;
and
a CDR3 comprising
(SEQ ID NO: 48)
GYDGFAY.

The heavy chain CDRs immediately above may be combined with a light chain comprising the following light chain CDRs:

a CDR1 comprising
(SEQ ID NO: 49)
RYSENIYSYLT;
a CDR2 comprising
(SEQ ID NO: 50)
NAKILAAGV;
and
a CDR3 comprising
(SEQ ID NO: 51)
QHHYDIPWT.

In some embodiments, the anti-Jagged1 antibody or antigen-binding fragment thereof from U.S. Pat. No. 9,725,518 used in the methods disclosed herein comprises a heavy chain comprising the following CDRs:

a CDR1 comprising
(SEQ ID NO: 52)
DYAMH;
a CDR2 comprising
(SEQ ID NO: 53)
NTYYGDARYNQKFKG;
and
a CDR3 comprising
(SEQ ID NO: 54)
GKEGFAY.

The heavy chain CDRs immediately above may be combined with a light chain comprising the following light chain CDRs:

a CDR1 comprising
(SEQ ID NO: 55)
RASENIYSYLA;
a CDR2 comprising
(SEQ ID NO: 56)
NAKTKAEVV;
and
a CDR3 comprising
(SEQ ID NO: 57)
QHHYGTPLT.

In other embodiments, the anti-Jagged1 antibody or antigen-binding fragment thereof used in the methods disclosed herein is an antibody or antigen-binding fragment thereof disclosed in U.S. Pat. No. 9,688,748 (the contents of which are incorporated by reference herein for that purpose).

In some embodiments, the anti-Jagged1 antibody or antigen-binding fragment thereof from U.S. Pat. No. 9,688,748 used in the methods disclosed herein comprises a heavy chain comprising the following CDRs:

a CDR1 comprising
(SEQ ID NO: 58)
SYAMS;
a CDR2 comprising
(SEQ ID NO: 59)
SIDPEGRQTYYADSVKG;
and
a CDR3 comprising
(SEQ ID NO: 60)
DIGGRSAFDY.

The heavy chain CDRs immediately above may be combined with a light chain comprising the following light chain CDRs:

a CDR1 comprising
(SEQ ID NO: 61)
RASQSISSY;
a CDR2 comprising
(SEQ ID NO: 62)
AASSLQS;
and
a CDR3 comprising
(SEQ ID NO: 63)
QQTVVAPPL.

In some embodiments, the anti-Jagged1 antibody is a commercially available antibody such as the αJag 1 ab10580 antibody from Abcam, Cambridge, U.K., http://www.abcam.com/ or the anti-Jagged1 antibody that is cat #sc-6011 from Santa Cruz Biotechnology, Santa Cruz, Calif.

In other embodiments, the antibody or antigen-binding fragment thereof used in the methods disclosed herein is the 15D11 monoclonal antibody used in the examples herein.

As disclosed in the examples, 15D11 displayed no detectable side effects in vivo while retaining a robust ability to inhibit Jagged1-mediated signaling in vitro and reduced in vivo bone metastasis of Jagged1-expressing breast cancer cells. In contrast to OPG-Fc, a decoy receptor for RANKL, 15D11 only inhibits pathological osteoclastogenesis but maintain a basal number of osteoclasts that is required for normal bone homeostasis. In addition to its inhibitory effect on bone metastasis of Jagged1-expressing tumor cells, 15D11 dramatically sensitizes bone metastasis to chemotherapy, which induces Jagged1 expression in osteoblasts to provide a survival niche to cancer cells.

The examples below demonstrate that various chemotherapy agents, including paclitaxel and cisplatin, induced Jagged1 expression in osteoblasts and mesenchymal stem cells (MSCs), which feeds back to tumor cells to activate Notch signaling and promote chemoresistance. Administration of 15D11 into an in vitro 3-D co-culture system or in various mouse models of bone metastasis significantly increased the sensitivity of tumor cells and bone metastasis to chemotherapy. Most strikingly, when chemotherapy was combined with 15D11, a nearly 100-fold reduction of bone metastasis burden was observed in the mouse model of bone metastasis.

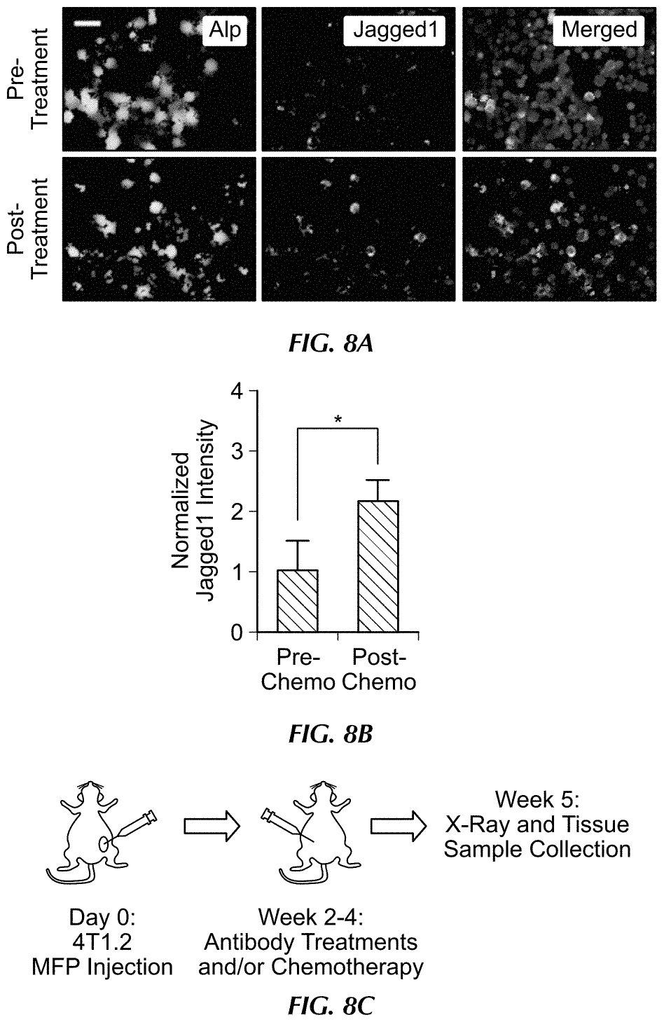

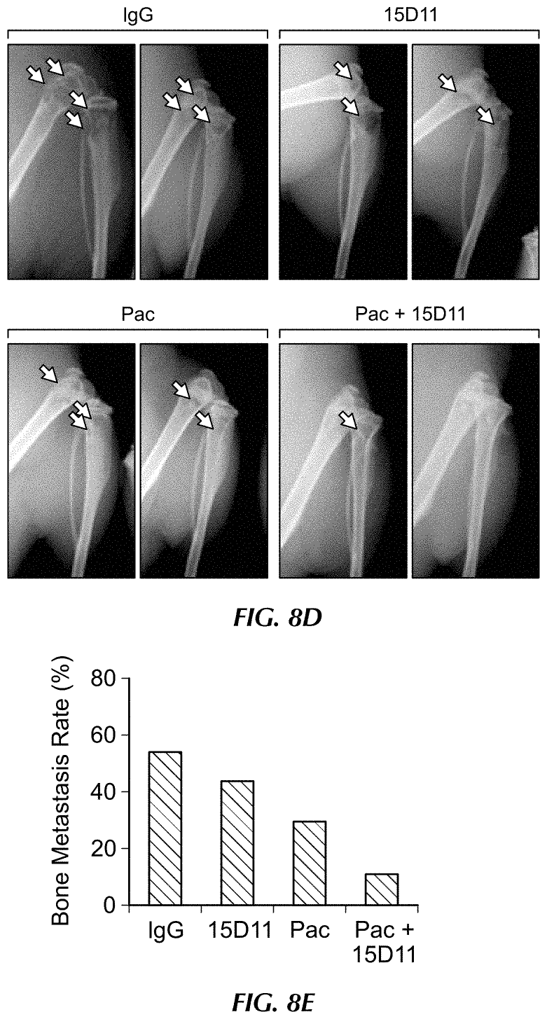

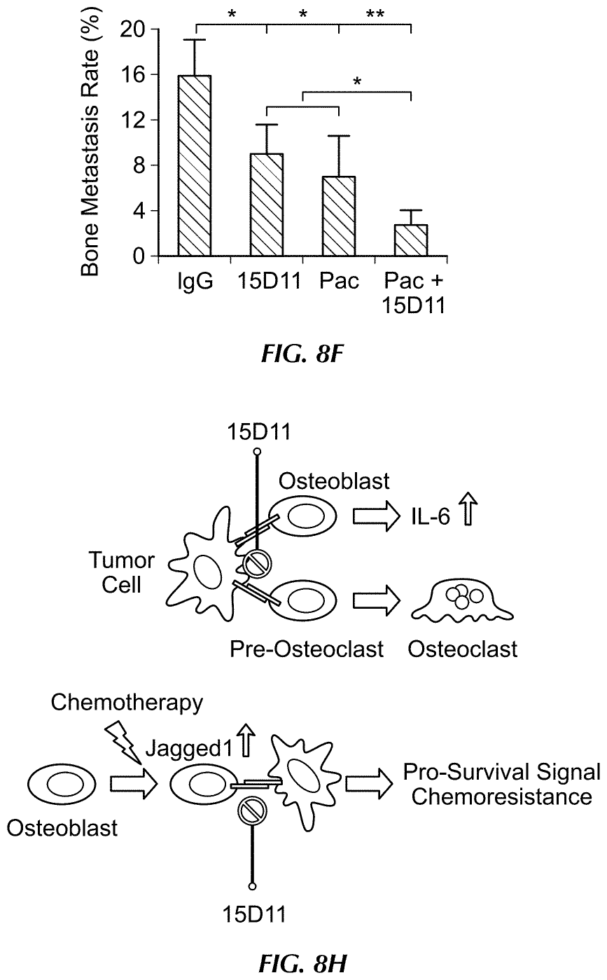

Thus, anti-Jagged1 antibodies represent unique therapeutic agents capable of targeting both tumor-derived Jagged1 and chemotherapy-induced Jagged1 in osteoblasts, thus inhibiting multiple downstream Notch signaling events that are important for both the expansion of osteolytic lesions and chemoresistance of cancer cells in bone (FIG. 8G).

In some embodiments, the anti-Jagged1 antibody or antigen-binding fragment thereof binds to an extracellular domain of Jagged1. In some embodiments, the anti-Jagged1 antibody or antigen-binding fragment thereof binds to a region comprising the DSL domain of Jagged1. In some embodiments, the anti-Jagged1 antibody or antigen-binding fragment thereof binds to a region comprising EGF1, EGF2 and/or EGF3 of Jagged1. In some embodiments, the Jagged1 is human Jagged1. In some embodiments, the anti-Jagged1 antibody or antigen-binding fragment thereof inhibits or interferes with binding of Jagged1 to a Notch receptor. In some embodiments, the anti-Jagged1 antibody or antigen-binding fragment thereof inhibits Notch signaling or Notch activation.

It is preferred that the anti-Jagged1 antibodies or antigen-binding fragments thereof disclosed herein include human constant domains when administered to a human. The antibodies can be any isotype or subtype, e.g., IgG1, IgG2a, IgG2b, IgG3, IgG4, IgM, IgA, IgD, or IgE. Effector functions may be optimized (e.g., to increase or reduce complement dependent cytotoxicity (CDC) or antibody dependent cellular cytotoxicity (ADCC) by choosing a particular class of antibody. The constant region (i.e., CHI, CH2, CH3, and/or the hinge region) may be modified in some embodiments in order to increase or decrease binding to an Fc or FcRn receptor, or to promote or stabilize heavy chain-heavy chain binding.

In some embodiments, the anti-Jagged1 antigen-binding fragment is an Fv fragment. An Fv is the smallest fragment that contains a complete heavy and light chain variable domain, including all six hypervariable loops (CDRs). Fv fragments lack constant domains and thus the variable domains are noncovalently associated.

In another embodiment, the anti-Jagged1 antigen-binding fragment is a single chain variable fragment (scFv). In an scFv, the heavy and light chains are connected into a single polypeptide chain by means of a linker (generally a peptide, e.g., (Gly-Gly-Gly-Gly-Ser)₃) (SEQ ID NO: 64) that allows the VH and VL domains to associate and form an antigen binding site. Since scFv fragments lack constant domains, they are considerably smaller than whole antibodies and lack the heavy-chain constant domain interactions with other biological molecules of full length antibodies.

Antibody fragments containing VH, VL, and optionally CL, CHI, or other constant domains may be prepared recombinantly and used in the methods disclosed herein to block Jagged1 activity. Also of use are other types of antibodies or antigen-binding fragments known in the art, e.g., diabodies, triabodies, single domain antibodies, and other monovalent and multivalent forms. For example, two single chain antibodies can be combined to form a diabody, i.e., a bivalent dimer. Each of the two chains of the diabody includes a VH domain connected to a VL domain by a short linker of about 5-10 amino acids.

Effector molecules which provide some desirable property (e.g., increased serum half-life) may be conjugated to the anti-Jagged1 antibody or antigen-binding portion thereof. An example of such an effector molecule is polyethyleneglycol (PEG). PEG may be attached to any amino acid side chain or terminal amino acid functional group, e.g., a free amino, imino, thiol, hydroxyl, or carboxyl group by methods that are well known in the art.

In some embodiments, the anti-Jagged1 antibody or antigen-binding fragment thereof has a dissociation constant (Kd) with respect to human Jagged1 of ≤1 μM, ≤100 nM, ≤10 nM, ≤1 nM, ≤0.1 nM, ≤0.01 nM, or ≤0.001 nM (10⁻⁸M or less, from 10⁻⁸M to 10⁻¹³M, or from 10⁻⁹ M to 10⁻¹³ M).

The anti-Jagged1 antibodies or antigen-binding fragments thereof are generally administered to patients combined with pharmaceutically acceptable carriers in the form of a pharmaceutical composition or formulation. Suitable pharmaceutically acceptable carriers include, for example, one or more of water, saline, phosphate buffered saline, dextrose, histidine, glutamate, citrate, mannitol, trehalose, sucrose, arginine, acetate, Polysorbate 80, Poloxamer 188, and the like, as well as combinations thereof. Pharmaceutically acceptable carriers may further comprise minor amounts of auxiliary substances such as wetting or emulsifying agents, preservatives, or buffers.

Pharmaceutically acceptable carriers, excipients, diluents, stabilizers, and/or other ingredients that may be used in formulations comprising anti-Jagged1 antibodies or antigen-binding fragments thereof can be found in standard references, e.g., Remington's Pharmaceutical Sciences, 16th edition, Osol, A. Ed. (1980). Acceptable carriers, excipients, or stabilizers are nontoxic at the dosages and concentrations employed. They may include buffers such as phosphate, citrate, and other organic acids; antioxidants such as ascorbic acid and methionine; preservatives such as octadecyldimethylbenzyl ammonium chloride, hexamethonium chloride, benzalkonium chloride, benzethonium chloride, as well as phenol, butyl or benzyl alcohol; alkyl parabens such as methyl or propyl paraben; catechol, resorcinol, cyclohexanol, 3-pentanol, and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates, including glucose, mannose, or dextrins; chelating agents such as EDTA; sugars such as sucrose, mannitol, trehalose, or sorbitol; salt-forming counter-ions such as sodium; metal complexes; and/or non-ionic surfactants such as TWEEN®, PLURONICS®, or polyethylene glycol (PEG).

In some embodiments, the anti-Jagged1 antibody is in the form of a sterile, preservative-free lyophilized powder for intravenous (IV) administration following reconstitution with water or other suitable diluent. In some embodiments, the lyophilized powder contains a single dose of 50-100 mg, 100-150 mg, 150-200 mg, 200-250 mg, 250-300 mg, 300-350 mg, 350-400 mg, 400-450 mg, or 450-500 mg of the anti-Jagged1 antibody.

In some embodiments, the anti-Jagged1 antibody may be formulated into a sustained-release preparation, e.g., by use of semipermeable matrices of solid hydrophobic polymers containing the antibody. Examples of sustained-release matrices include polyesters, hydro gels (for example, poly(2-hydroxyethyl-methacrylate), or poly(vinylalcohol)), polylactides, copolymers of L-glutamic acid and γ-ethyl-L-glutamate, nondegradable ethylene-vinyl acetate, degradable lactic acid-glycolic acid copolymers, and poly-D-(−)-3-hydroxybutyric acid.

The amount and/or concentration of anti-Jagged1 antibody or antigen-binding fragment thereof in the pharmaceutical compositions or formulations may vary and will depend on factors such as: the disease state, the age, sex, general health, etc. of the patient.

For example, the pharmaceutical compositions or formulations may comprise, in a single dose, anti-Jagged1 antibody thereof in various amounts, e.g., 10 mg/ml to 500 mg/ml, 25 mg/ml to 300 mg/ml, 50 mg/ml to 200 mg/ml, 75 mg/ml to 150 mg/ml, or 100 mg/ml to 125 mg/ml. The compositions also may comprise anti-Jagged1 antibody at about 10 mg/ml, 20 mg/ml, 30 mg/ml, 40 mg/ml, 50 mg/ml, 60 mg/ml, 70 mg/ml, 80 mg/ml, 90 mg/ml, 100 mg/ml, 110 mg/ml, 120 mg/ml, 130 mg/ml, 140 mg/ml, or 150 mg/ml. Other concentrations are also possible. Corresponding values for anti-Jagged1 antigen binding fragments may be readily appreciated by those skilled in the art, depending on the type of fragment.

In certain embodiments, the anti-Jagged1 antibody or antigen-binding fragment thereof is administered to the patient by intravenous infusion, i.e., by introduction of the anti-Jagged1 antibody or antigen-binding fragment thereof into the vein of a the patient over a certain period of time, e.g., about 2 minutes, about 5 minutes, about 10 minutes, about 20 minutes, about 30 minutes, about 1 hour, about 2 hours, about 3 hours, about 4 hours, about 5 hours, about 6 hours, about 7 hours, or about 8 hours, or longer.

In certain embodiments, the anti-Jagged1 antibody or antigen-binding fragment thereof is administered to the patient subcutaneously, i.e., under the skin of the patient.

In certain embodiments, a single dose of the anti-Jagged1 antibody or antigen-binding fragment thereof is administered to a patient every day, every other day, every third day, once a week, twice a week, three times a week, once every two weeks, or once a month. In other embodiments, two, three or four doses are administered to a patient every day, every other day, every third day, once a week, once every two weeks, or once a month. In some embodiments, a dose is administered for 2 consecutive days, 3 consecutive days, 4 consecutive days, 5 consecutive days, 6 consecutive days, 7 consecutive days, 14 consecutive days, or 21 consecutive days. In certain embodiments, a dose of a compound or a composition is administered each day for 1 month, 1.5 months, 2 months, 2.5 months, 3 months, 4 months, 5 months, 6 months or more.

Methods of administration also include, but are not limited to, parenteral, intradermal, intravitrial, intraperitoneal, intranasal, epidural, oral, sublingual, intracerebral, intravaginal, transmucosal, rectally, by inhalation, or topically, particularly to the ears, nose, eyes, or skin. The mode of administration may be left to the discretion of the practitioner.

Local administration may be desirable in some circumstances. This may be achieved, e.g., by local infusion, topical application, injection, by means of a catheter, or by means of an implant. Such methods of administration may be used to selectively target bone metastases without substantial release of therapeutic agent into the bloodstream.

Pulmonary administration may also be useful, e.g., by use of an inhaler or nebulizer, and a formulation with an aerosolizing agent. Liposomal delivery or controlled release delivery may also be employed in some embodiments.

A wide variety of chemotherapeutic agents may be combined with anti-Jagged1 antibodies or antigen-binding fragments thereof in the methods disclosed herein. Types of chemotherapeutic agents that may be used include alkylating agents, alkyl sulfonates, aziridines, ethylenimines, methylamelamines, colchicines, camptothecins, nitrogen mustards, nitrosoureas, plant alkaloids, bisphosphonates, anthracyclines, anti-metabolites, anti-microtubule agents, topoisomerase inhibitors, cytotoxic antibiotics, metal salts, toxoids, taxanes, pyrimidine analogs, purine analogs, aromatase inhibitors, mitomycins, androgens, anti-adrenals, folic acid replenishers, anti-folates, dihydrofolate reductase inhibitors, thymidylate synthase inhibitors, vinca alkaloids, anti-hormonal agents, and pharmaceutically acceptable salts, acids, or derivatives of any of the above, as well as combinations of two or more of the above.

Specific chemotherapeutic agents that may be used include TAXOL® (paclitaxel), docetaxel, ADRIAMYCIN® (doxorubicin), epirubicin, 5-fluorouracil, CYTOXAN® (cyclophosphamide), carboplatin, PLATINOL® (cisplatin), IBRANCE® (palbociclib), ARIMIDEX® (anastrozole), AVASTIN® (bevacizumab), XELODA® (capecitabine), DOXIL® (doxorubicin liposomal injection), AROMASIN® (exemestane), GEMZAR® (gemcitabine), IXEMPRA® (ixabepilone), FEMARA® (letrozole), or HERCEPTIN® (trastuzumab).

In some embodiments, the combination treatments of anti-Jagged1 antibody or antigen-binding fragment thereof plus chemotherapeutic agent disclosed herein extend progression free survival (PFS) or overall survival (OS) of the patient in a synergistic manner, i.e., by at least about 5%, at least about 10%, at least about 15%, at least about 20%, at least about 25% more, at least about 50% more, at least about 100% more, at least about 200% more, at least about 300% more, at least about 500% more, or at least about 1,000% more, than the combined PFS or OS achieved by the individual therapeutic substances of the combination treatments when administered alone.

In some embodiments, the combination treatments of anti-Jagged1 antibody or antigen-binding fragment thereof plus chemotherapeutic agent disclosed herein are used, together with surgery, as a treatment for breast cancer. That is, the combination treatment is administered shortly before surgery, shortly after surgery, in the same time frame before or after surgery that chemotherapy alone is conventionally administered to treat breast cancer. In some embodiments, the combination treatments and surgery are used as a first line treatment for breast cancer, i.e., before any other treatments for the breast cancer have been used.

In some embodiments, the combination treatments of anti-Jagged1 antibody or antigen-binding fragment thereof plus chemotherapeutic agent disclosed herein are used as second line therapy for breast cancer, i.e., after another treatment has been used. In some embodiments, the combination treatment of anti-Jagged1 antibody or antigen-binding fragment thereof plus chemotherapeutic agent disclosed herein is used as third line therapy for breast cancer, i.e., after two other treatments have been used.

In some embodiments, the combination treatments of anti-Jagged1 antibody or antigen-binding fragment thereof plus chemotherapeutic agent disclosed herein are used as a treatment for triple negative breast cancer.

In some embodiments, the combination treatments of anti-Jagged1 antibody or antigen-binding fragment thereof plus chemotherapeutic agent disclosed herein are used to treat breast cancer before the breast cancer has metastasized and the treatment prevents or delays the metastasis of the breast cancer.

In some embodiments, the combination treatment of anti-Jagged1 antibody or antigen-binding fragment thereof plus chemotherapeutic agent disclosed herein is used to treat breast cancer before the breast cancer has been detected as having metastasized.

In some embodiments, the combination treatment of anti-Jagged1 antibody or antigen-binding fragment thereof plus chemotherapeutic agent disclosed herein is used to treat breast cancer after the breast cancer has metastasized.

Synergism between the anti-Jagged1 antibody or antigen-binding fragment thereof and chemotherapeutic agent permits the medical practitioner to administer lower doses of chemotherapeutic agent than usual to a patient, yet still achieve the same, or better, therapeutic effect. For example, a typical beginning dose schedule for paclitaxel in the treatment of metastatic breast cancer in human females might be 175 mg/m² of body surface area by intravenous infusion for 3 hours weekly or every 3 weeks for four doses. Combining paclitaxel with an anti-Jagged1 antibody or antigen-binding fragment thereof is expected to permit the use of lower amount of paclitaxel.

Accordingly, disclosed herein is a method of treating breast cancer in a human female comprising administering to a human female in need thereof a therapeutically effective amount of an anti-Jagged1 antibody or antigen-binding fragment thereof and paclitaxel at a dose of 70-160 mg/m², 80-150 mg/m², 90-130 mg/m², or 100-125 mg/m² of body surface area.

Also disclosed herein is a method of treating breast cancer in a human female comprising administering to a human female in need thereof a therapeutically effective amount of an anti-Jagged1 antibody or antigen-binding fragment thereof and paclitaxel at a dose of 70-80 mg/m², 80-90 mg/m², 90-100 mg/m², 100-110 mg/m², 110-120 mg/m², 120-130 mg/m², 130-140 mg/m², or 140-150 mg/m², of body surface area.

Similarly, synergy between the anti-Jagged1 antibody or antigen-binding fragment thereof and doxorubicin permits lower than usual doses of doxorubicin to be used. Thus, disclosed herein is a method of treating breast cancer in a human female comprising administering to a human female in need thereof a therapeutically effective amount of an anti-Jagged1 antibody or antigen-binding fragment thereof and doxorubicin at a dose of 20-30 mg/m², 30-40 mg/m², or 40-50 mg/m² of body surface area.

Cyclophosphamide and docetaxel may also be administered at lower than usual doses. Disclosed herein is a method of treating breast cancer in a human female comprising administering to a human female in need thereof a therapeutically effective amount of an anti-Jagged1 antibody or antigen-binding fragment thereof and cyclophosphamide at a dose of 250-300 mg/m², 300-350 mg/m², or 350-400 mg/m² of body surface area. Disclosed herein is a method of treating breast cancer in a human female comprising administering to a human female in need thereof a therapeutically effective amount of an anti-Jagged1 antibody or antigen-binding fragment thereof and docetaxel at a dose of 35-40 mg/m², 40-45 mg/m², or 45-50 mg/m² of body surface area.

### EXAMPLES

### Example 1

### Generation and Characterization of a Fully Human Monoclonal Antibody Against Jagged1

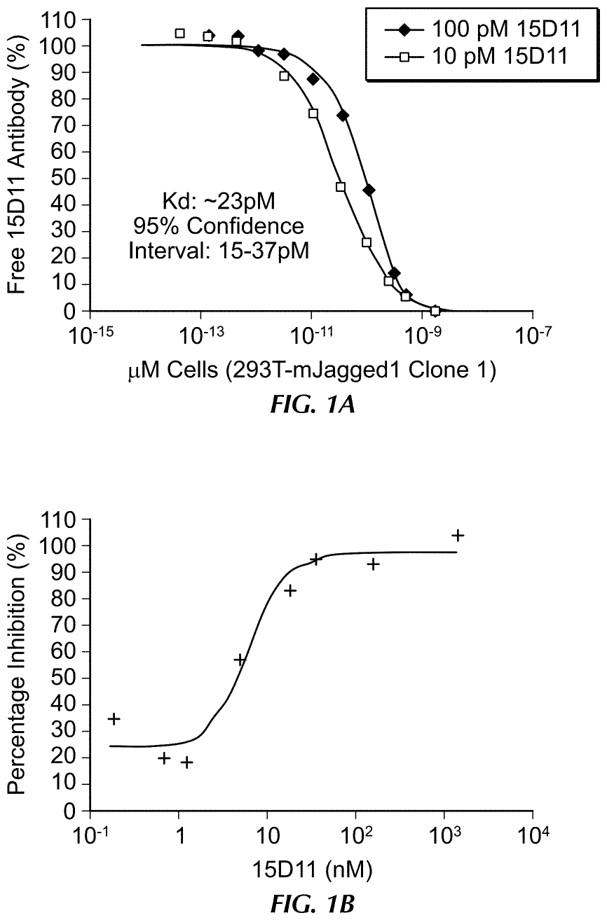

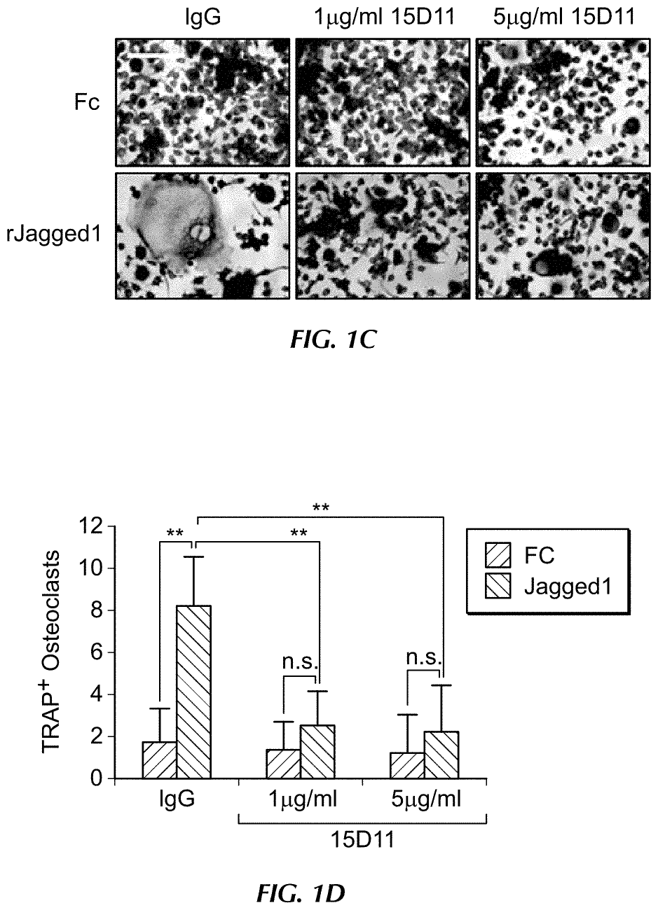

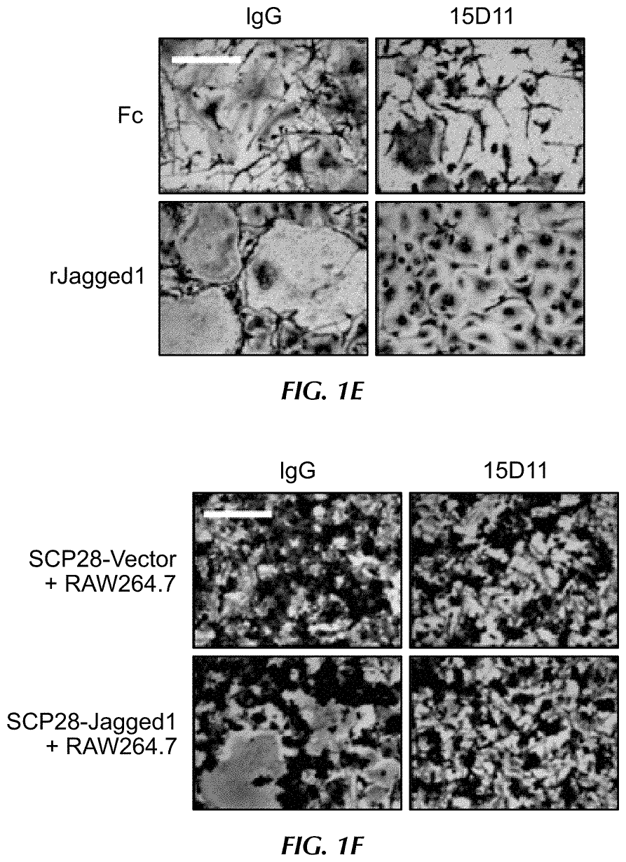

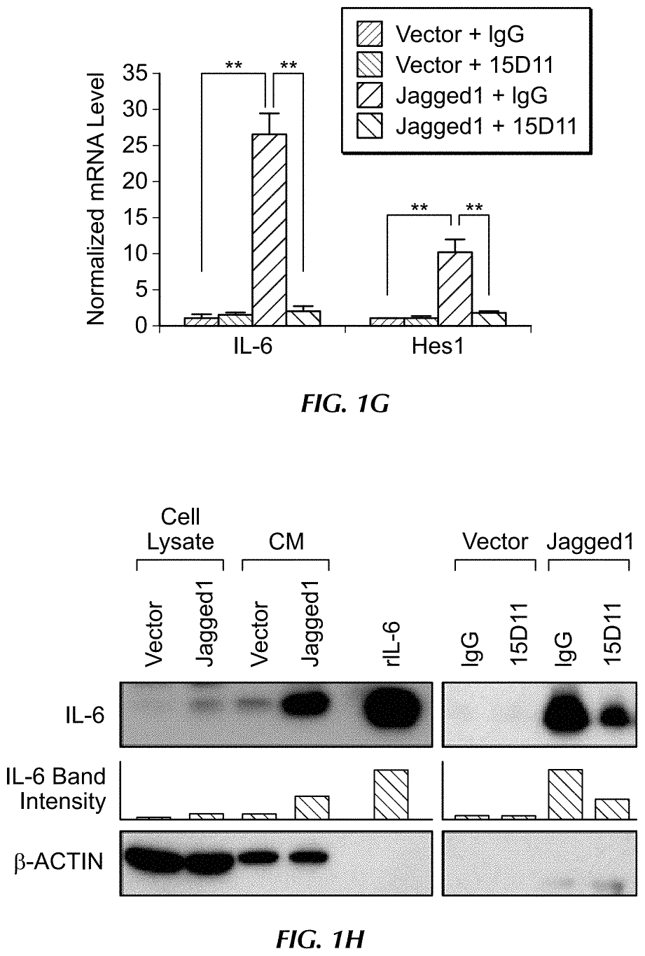

The XenoMouse® technology was used to generate fully human monoclonal antibodies against Jagged1. These mice have the endogenous mouse immunoglobulin loci inactivated and large transgenes introduced that are capable of recombination and fully human antibody repertoire development (Mendez et al., 1997). In brief, XenoMouse animals, including strains XMG2KL and XMG4KL (Kellermann and Green, 2002), were immunized with Chinese hamster cells (CHO) cells transiently expressing human Jagged1 to generate large panels of antibodies. Cell based fluorometric microvolume assay technology (FMAT) was used to analyze the specific binding of these antibodies to cell surface human Jagged1 protein. These antibodies were then counter screened to exclude cross-reactive binding to other major Notch ligands, including human Jagged2 and Dll4. Only the antibodies with selective binding to Jagged1 were then further assayed to identify those capable of blocking the binding of Jagged1 to Notch 1-3 receptors. In order to identify a suitable antibody antagonist that could be used for murine in vivo studies, the most potent blockers of the Notch receptor interaction were then further assayed for cross-reactive binding to murine Jagged1. The antibody 15D11 was identified through these processes. 15D11 is of particularly high affinity to the murine Jagged1 protein with an approximate dissociation constant (Kd) of 23 pM in the enzyme kinetic exclusion assay (KinExA) (FIG. 1A). Using a Notch luciferase reporter assay, it was determined that 15D11 has a half maximal inhibitory concentration (IC50) of 3.63 nM to human Jagged1 and 14.07 nM to murine Jagged1 (FIG. 1B).

When administered in vivo (10 mg/kg, twice a week), 15D11 displayed minimal general toxicity based on body weight measurement, in contrast to significant weight loss of mice treated with γ-secretase inhibitor (GSI). Haematoxylin and eosin staining (H&E) and Alcian blue staining revealed no obvious GI track toxicity after 15D11 treatment, compared to significant goblet cell metaplasia seen in GSI-treated animals. To analyze liver toxicity, the activity of alanine aminotransferase (ALT) and aspartate aminotransferase (AST) in the sera of these mice after the respective drug treatments was measured. As a positive control, CCl4 treatment induced dramatic increase of ALT and AST activity, while no change was observed after 15D11 or GSI treatment. A complete blood count (CBC) test further indicated no significant hematologic toxicity of 15D11. Analysis of T cell population and activation status also did not reveal any difference between the IgG or 15D11 treatment groups. Taken together, these results indicate that 15D11 is a specific Jagged1-targeting agent with excellent safety profile for in vivo application.

### Example 2

### Effect of Anti-Jagged1 Antibody on Jagged1-Dependent Osteoclastogenesis

An in vitro assay was developed to test the potential inhibitory effect of 15D11 on Jagged1-dependent osteoclastogenesis. RAW264.7, a monocyte/macrophage cell line with the ability to differentiate into osteoclasts, was seeded on either Fc-coated or recombinant Jagged1 (rJagged1) coated plates with low level of RANKL (5 ng/ml), a concentration that is not sufficient to induce osteoclast differentiation (Ell et al., 2013). Within 5 days, large multi-nuclear mature osteoclasts were generated on rJagged1-coated plates, but only limited numbers were seen on Fc-coated plates. Furthermore, such Jagged1-dependent osteoclast differentiation was completely blocked by 15D11 (FIGS. 1C and 1D). Similar result was observed when using bone marrow-derived primary pre-osteoclasts in the assay (FIG. 1E). To more closely mimic osteoclastogenesis induced by tumor-derived Jagged1, RAW264.7 cells were co-cultured with SCP28, a MDA-MB-231 derivative cell line with moderate bone metastatic ability that had been stably labeled with luciferase and GFP (Kang et al., 2003), with or without stable overexpression of Jagged1. Administration of 15D11 also inhibited osteoclast differentiation in this co-culture assay (FIG. 1F).

### Example 3

### Effect of Anti-Jagged1 Antibody on Jagged1-Notch Signaling Activities in Osteoblasts

Another previously reported mechanism for tumor-derived Jagged1 to promote bone metastasis is through increasing IL-6 production from osteoblasts (Sethi et al., 2011). As previously reported, co-culture of SCP28 with the mouse MC3T3 osteoblast cell line led to elevated expression of IL-6 in a Jagged1-dependent manner (FIGS. 1G and 1H). Administration of 15D11 in tumor-osteoblast co-culture significantly repressed the expression of the Notch induced gene Hes1 (detected by species-specific qRT-PCR) (FIG. 1G), as well as IL-6 expression at both the mRNA and protein levels (FIGS. 1G and 1H). Taken together, these in vitro experiments confirm that 15D11 effectively blocks Jagged1-Notch signaling activities that are important for bone metastasis development.

### Example 4

### Anti-Jagged1 Antibody Attenuates Bone Metastasis of Jagged1-Expressing Tumor Cells

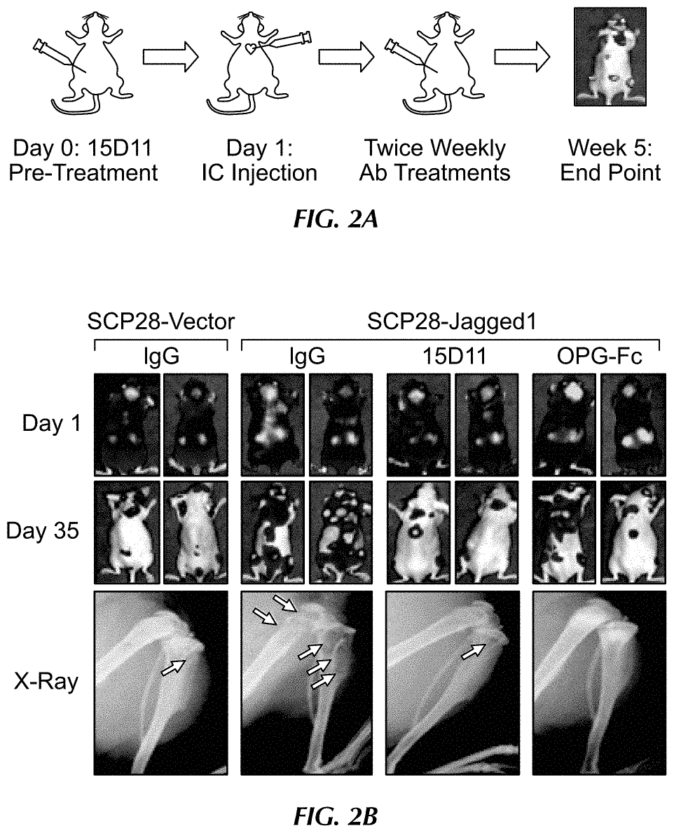

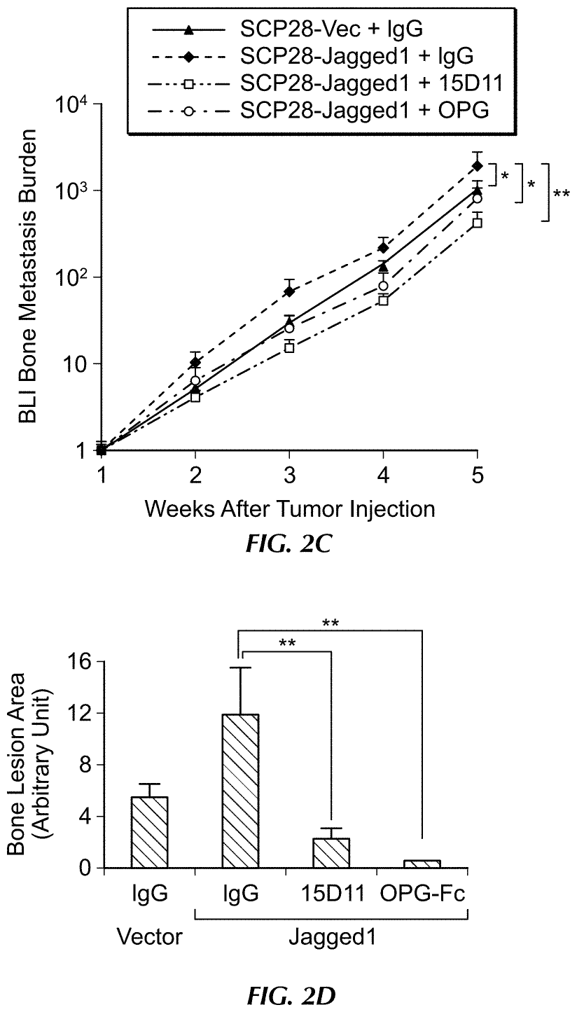

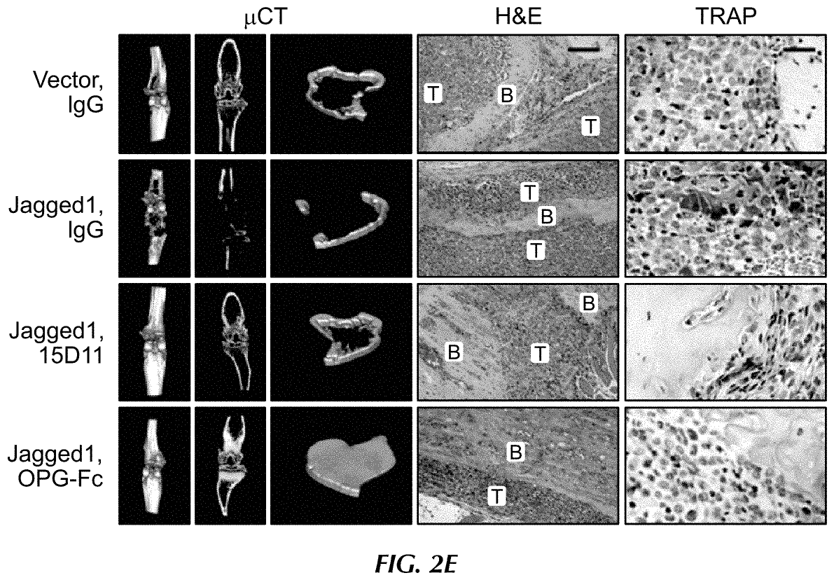

To evaluate the therapeutic effect of 15D11 on bone metastasis, a well-established xenograft model for bone metastasis involving SCP28 cells was used. SCP28 has a low basal level of Jagged1 expression, and ectopic overexpression of Jagged1 in SCP28 has been demonstrated to enhance its bone metastatic ability (Sethi et al., 2011). Tumor cells were inoculated via intracardiac (IC) injection into athymic nude mice to generate bone metastasis. 15D11 antibody treatment was initiated one day before tumor cell injection and the treatment continued twice a week. Bone metastasis was monitored by weekly bioluminescence imaging (BLI) until mice were euthanized at the end of the experiment (˜5 weeks) for histopathological analysis (FIG. 2A). The decoy receptor for RANKL, OPG-Fc (equivalent to clinically approved RANKL blocking antibody denosumab), was also to block osteoclast differentiation as the positive control in the experiment. Jagged1 overexpression led to a significant increase in bone metastasis burden (FIGS. 2B and 2C), consistent with a previous report (Sethi et al., 2011). BLI analysis revealed a ˜5 fold decrease in SCP28-Jagged1 bone metastasis in mice treated with 15D11 compared to IgG control group. As expected, OPG-Fc treatment also decreased the bone metastasis burden of mice (FIGS. 2B and 2C). X-ray imaging of the hind limbs of mice injected with SCP28-Jagged1 revealed significantly more osteolytic areas compared to mice injected with the control SCP28 cells, a phenotype that can be reversed by 15D11 treatment (FIGS. 2D and 2E). While OPG-Fc treatment also protected bone from osteolysis, it also led to an abnormal increase of the trabecular bone density as observed by X-ray and microcomputer tomography (μCT) analysis (FIG. 2D-E). This is consistent with previous observations of animals treated with osteoclast targeting agents such as OPG-Fc (Bargman et al., 2012) and bisphosphonates (Korpal et al., 2009). Consistently, tartrate-resistant acid phosphatase (TRAP) immunostaining of bone sections indicated an almost complete absence of osteoclasts in mice treated with OPG-Fc. Interestingly, while Jagged1 overexpressing SCP28 promoted the recruitment of mature osteoclasts in the bone lesions, a considerable number of osteoclasts can still be observed in mice treated with 15D11 (FIG. 2E). Importantly, there was no abnormal increase of trabecular bone density in 15D11-treated animals, in contrast to OPG-Fc treated mice (FIG. 2E). Taken together, these results suggest that 15D11 inhibits pathological activation of osteoclasts by tumor-derived Jagged1, but spares normal osteoclast activities needed to maintain normal bone homeostasis.

### Example 5

### Effect of Anti-Jagged1 Antibody on Established Bone Metastasis

To test the therapeutic effect of 15D11 on established bone metastasis, the SCP2 cell line, an extremely aggressive bone metastatic variant of MDA-MB-231 with a high level of endogenous Jagged1 expression (Sethi et al., 2011), was used. The SCP2 cells rapidly generated multiple osteolytic bone metastases within 1 week after IC injection into nude mice, which often results in mortality within 4 weeks. We initiated twice weekly injection of 15D11 one week after IC injection of SCP2 cells when bone metastases were well established. In this model of late treatment of aggressive bone metastasis, a trend of reduced bone metastasis by either 15D11 or OPG-Fc treatment alone compared to the control group was detected, although neither trend reached statistical significance at the end point of the experiment (4 weeks) when the mice succumbed to metastatic cancers. This is similar to the lack of reduced metastatic tumor burden in late treatment protocols using other bone metastasis targeting agents, such as bisphosphonates or TGF-β receptor I inhibitor (Korpal et al., 2009). Combinatory treatment of 15D11 and OPG-Fc resulted in a significant ˜5-fold decrease of bone metastasis 3 weeks after the initiation of treatments, indicating the benefit of targeting two different molecular mediators of bone metastasis. Similar to the preventive treatment protocol, 15D11 treatment reduced the number of osteoclasts, although some basal level of osteoclasts can still be observed, as compared to the near complete absence of osteoclasts in mice treated with OPG-Fc.

### Example 6

### Anti-Jagged1 Antibody Sensitizes Bone Metastases to Chemotherapy

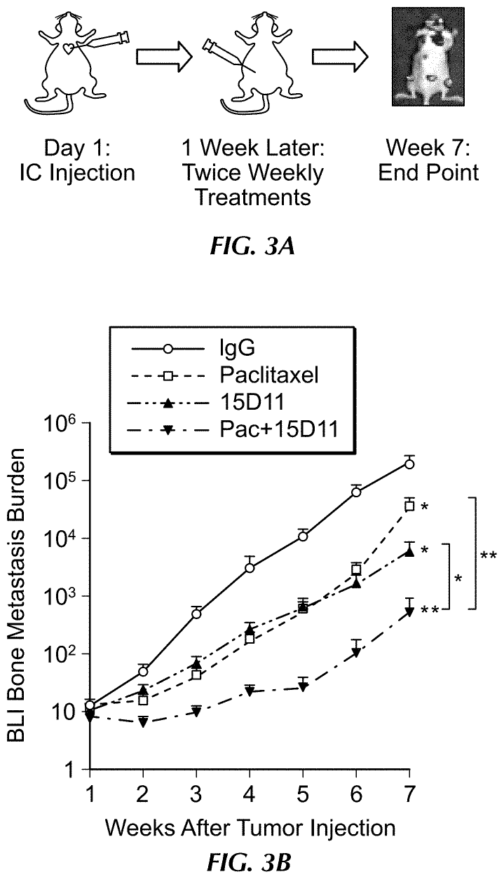

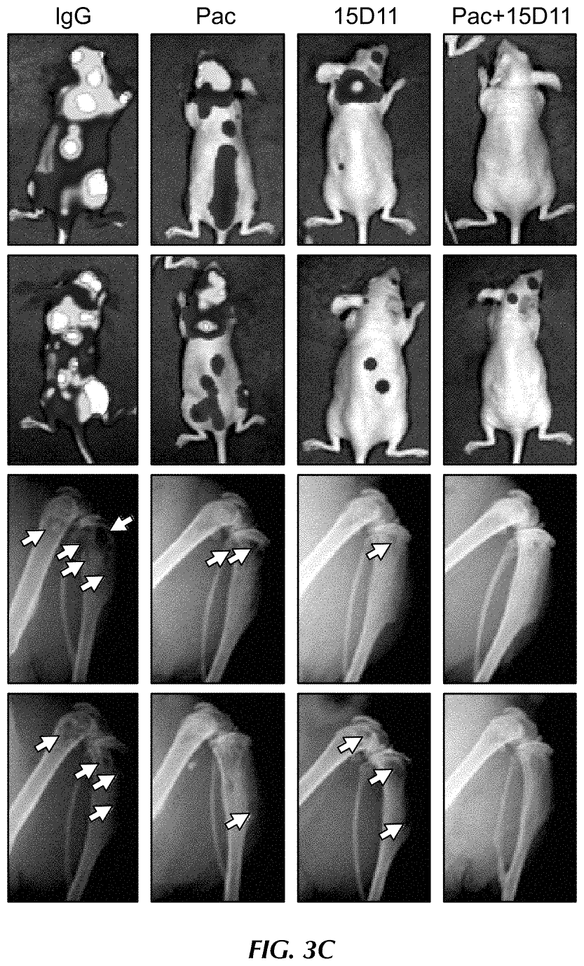

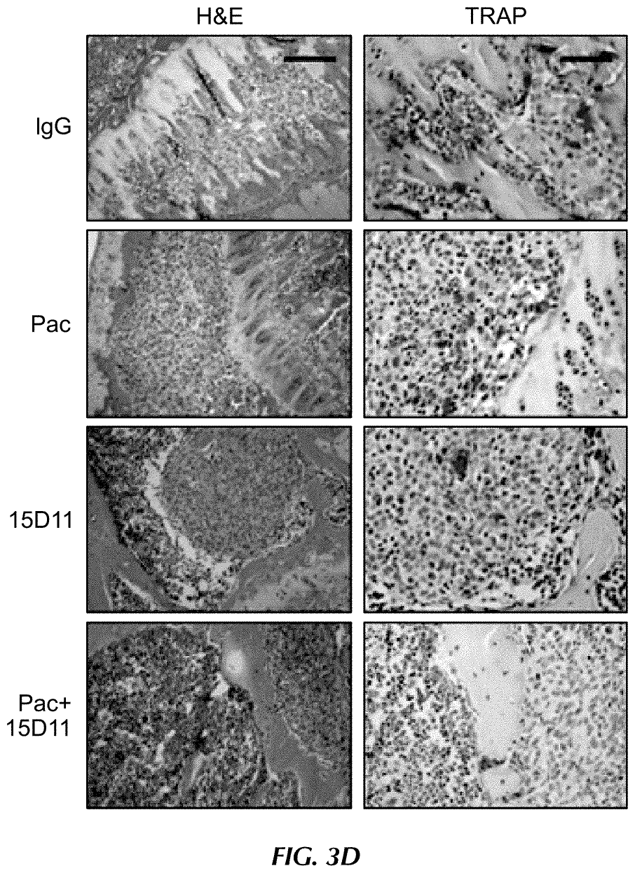

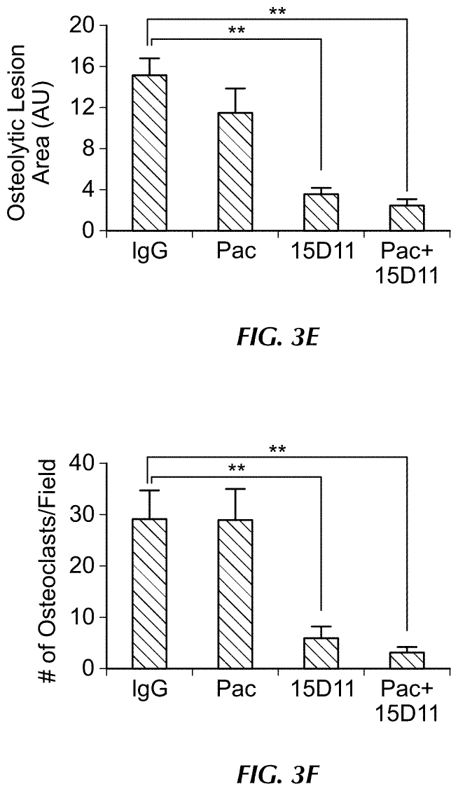

Chemotherapy is commonly used to manage bone metastasis, although bone lesions are usually more refractory to chemotherapy (Gu et al., 2004). To evaluate the combinatorial effect of 15D11 with conventional chemotherapy, we treated bone metastasis generated by SCP28-Jagged1 cells with 15D11 and paclitaxel, using the late treatment protocol (treatment initiated 1 week after IC injection) (FIG. 3A). The mouse group injected with SCP28-Jagged1 progressed slowly to the moribund state compared to mouse group injected SCP2 (7 weeks compared to 4 weeks with SCP2), which provided us with a sufficient time to observe the therapeutic effect of single or combinatory treatment of 15D11 and paclitaxel. Mice were treated with IgG (as negative control), 15D11, paclitaxel, or both, one week after IC injection of SCP28-Jagged1 to establish bone metastases. Monotherapy using 15D11 resulted in a nearly 10-fold reduction of metastasis burden (FIG. 3B). While paclitaxel alone significantly slowed down bone metastasis progression at early time points, resistance to chemotherapy emerged at later time points, resulting in only a mild reduction of tumor burden at the end point of the experiment (FIG. 3B). Strikingly, when mice were treated with both 15D11 and paclitaxel, a synergistic >100-fold reduction of bone metastasis was observed compared to the IgG control group (FIGS. 3B and 3C). X-ray images and TRAP staining for osteoclasts confirmed significant decrease of osteoclast number and osteolytic lesions after 15D11 treatment alone or combined with chemotherapy (FIG. 3C-F). In particular, there is almost no osteolytic bone area in the combined treatment group, consistent with the BLI data. Taken together, these results demonstrate a potent synergistic effect of combined treatment of paclitaxel and 15D11 in diminishing bone metastasis generated by SCP28-Jagged1.

### Example 7

### Synergistic Effect of Anti-Jagged1 Antibody and Chemotherapy

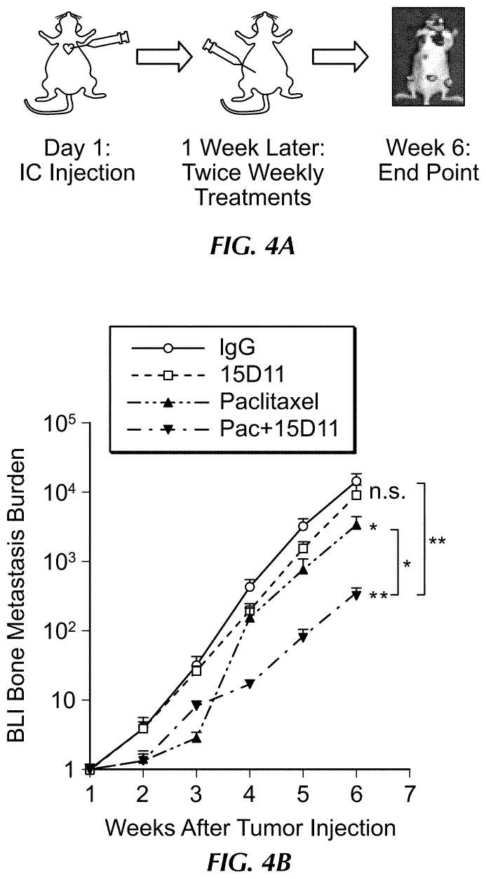

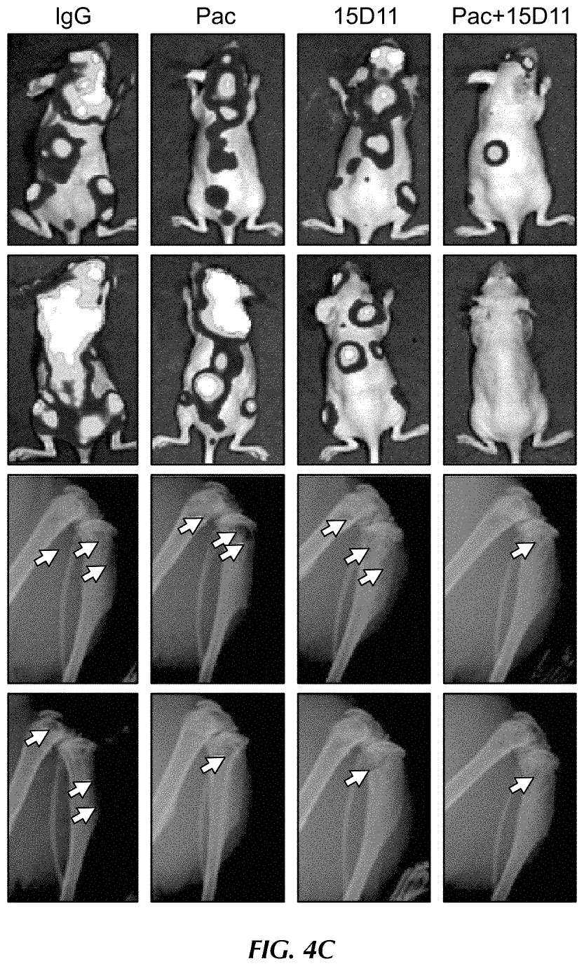

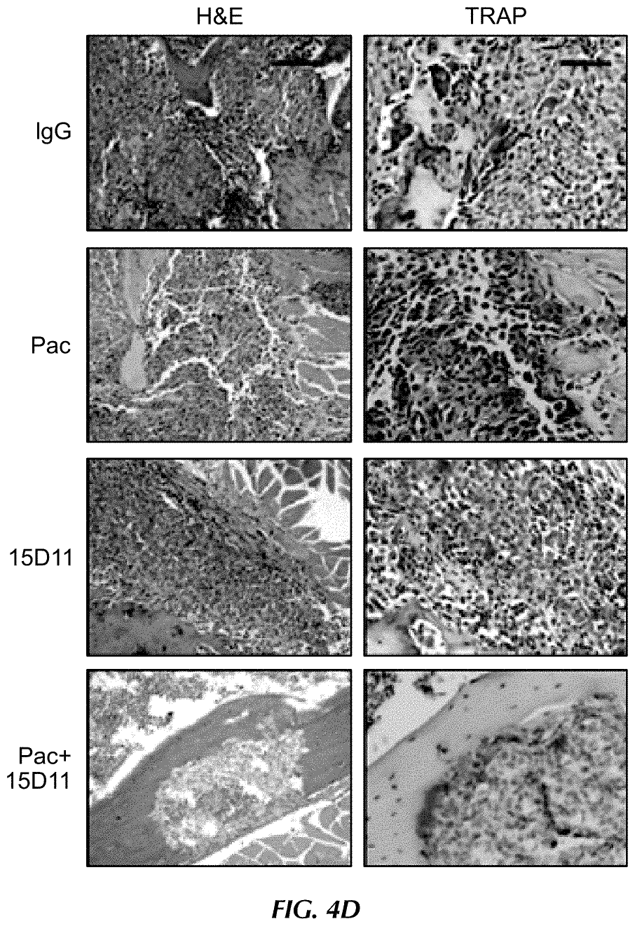

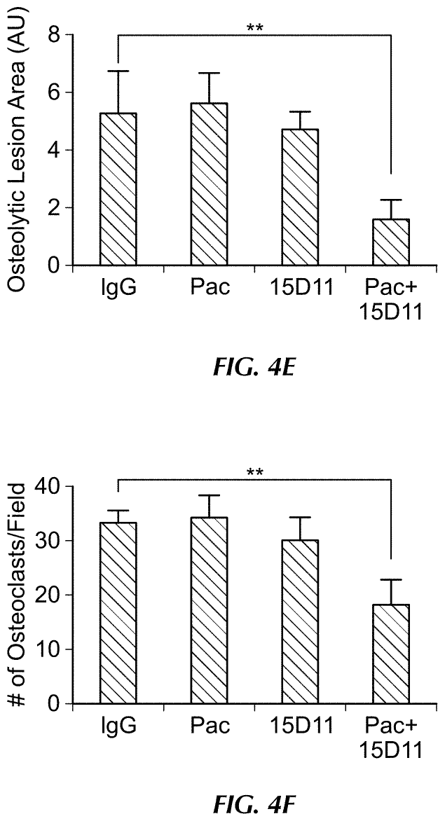

The strong inhibitory effect of the combined treatment on bone metastasis could potentially be explained by the combined effect of blocking Jagged1-dependent stromal engagement by 15D11 and cytotoxicity of paclitaxel to tumor cells. If this is indeed the case, no synergistic therapeutic benefit should be expected when chemotherapy is combined with 15D11 for the treatment of bone metastasis generated by tumor cells with low or no Jagged1 expression. We thus used the parental SCP28 cells, which has very low endogenous Jagged1 level, to test this directly. The same bone metastasis and treatment experiments as we did with SCP28-Jagged1 were performed (FIG. 4A). Paclitaxel initially reduced bone metastasis burden, but the lesions quickly become refractory to chemotherapy and only slightly reduced metastatic burden (FIGS. 4B and 4C). As expected from the lack of Jagged1 expression in SCP28, 15D11 had no effect on reducing bone metastasis burden by SCP28 (FIGS. 4B and 4C). To our surprise, the combined treatment group displayed a dramatic reduction of bone metastasis burden, decreased osteolytic bone lesion areas, and reduced osteoclast number (FIG. 4B-F). Such a therapeutic response is far superior than single treatment of either paclitaxel or 15D11 alone, suggesting a synergy of targeting Jagged1 along with chemotherapy for bone metastasis even in in tumor cells with low level of Jagged1 expression.

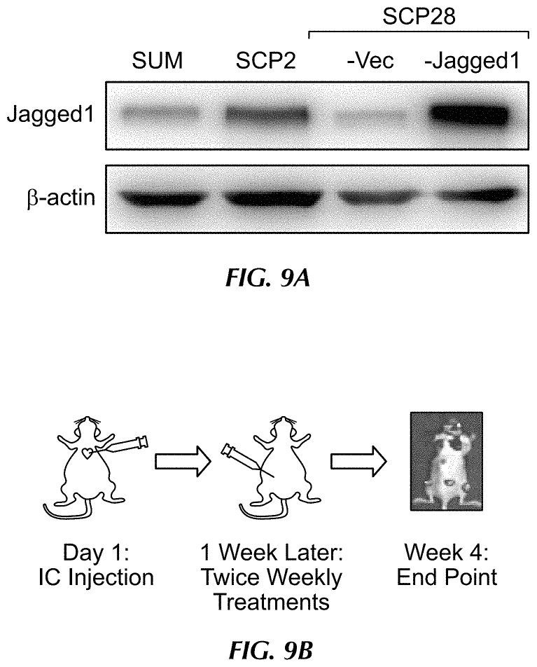

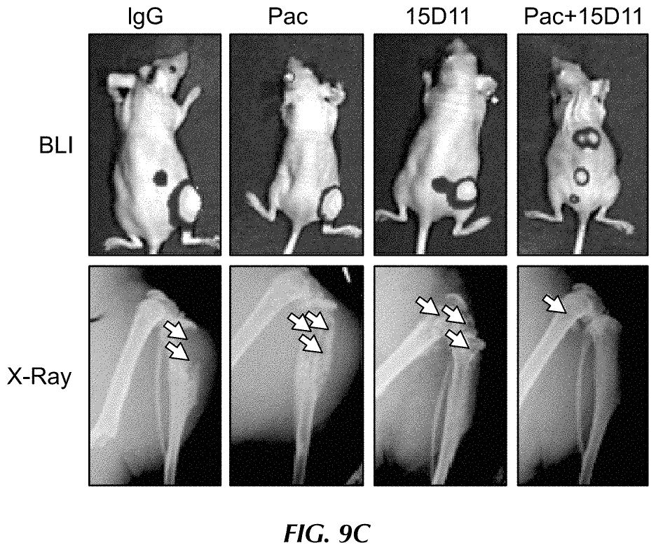

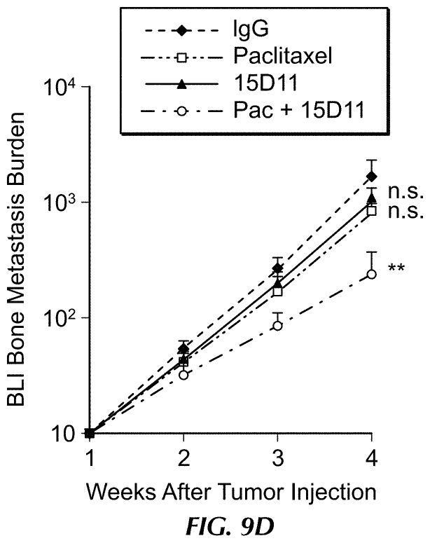

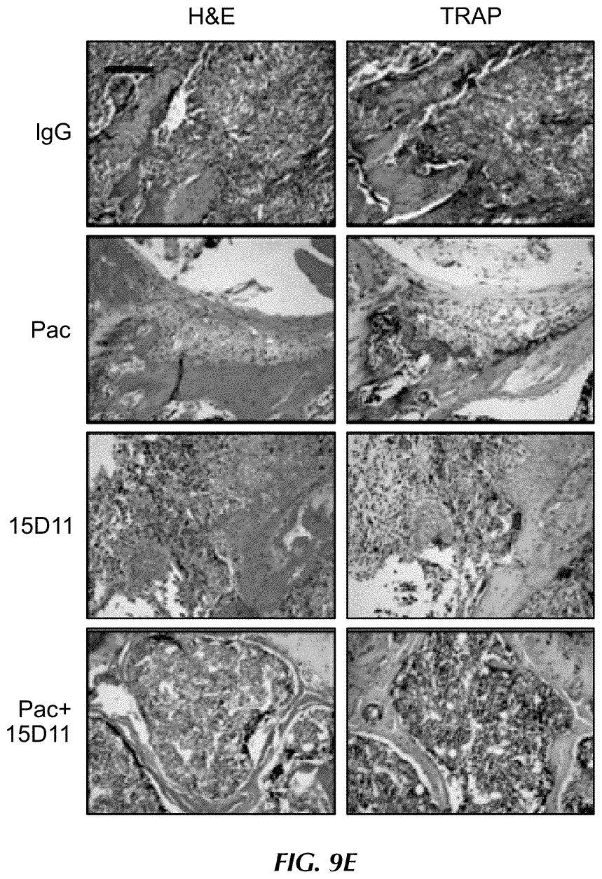

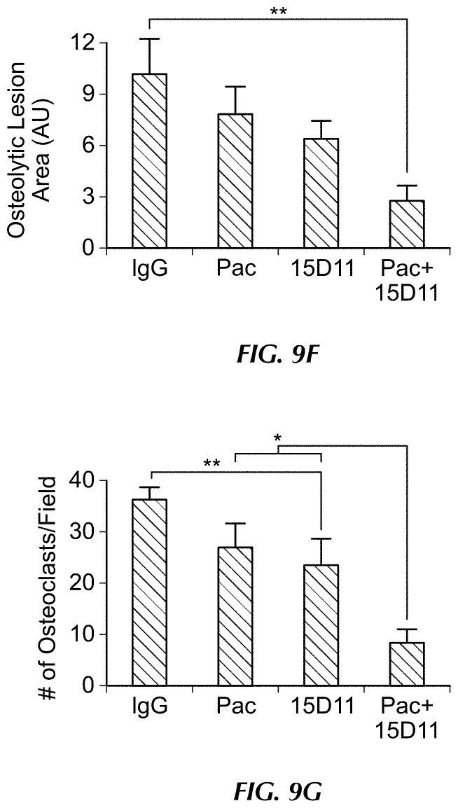

To validate whether the observed synergy of combined treatment can also be observed in other models of bone metastasis beyond the MDA-MB-231 series, the bone metastatic SUM1315-M1B1 cell line was used. SUM1315-M1B1 cells have been recently developed in the inventors' laboratory by in vivo selection for increased bone metastatic propensity from the parental SUM1315 breast cancer cell line (Forozan et al., 1999), as a metastatic model of breast cancer. SUM1315-M1B1 has a similarly low basal level of Jagged1 expression as SCP28. Again, a significant reduction of bone metastasis burden, osteolytic lesion area, and osteoclast numbers was only observed in mice treated with both paclitaxel and 15D11 (FIG. 9B-G). Taken together, we identified a potent synergistic inhibitory effect on breast cancer bone metastasis by combining chemotherapy and 15D11, and such therapeutic synergy is not dependent on high expression level of Jagged1 in tumor cells.

### Example 8

### Chemotherapy Induces Jagged1 Expression in Osteoblast Lineage Cells

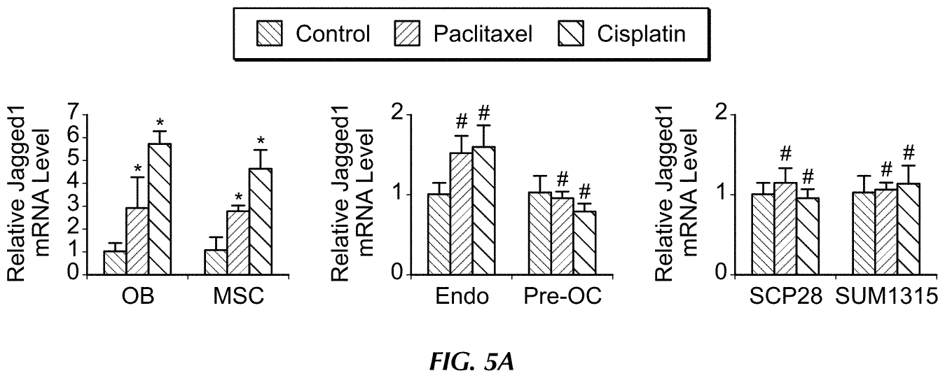

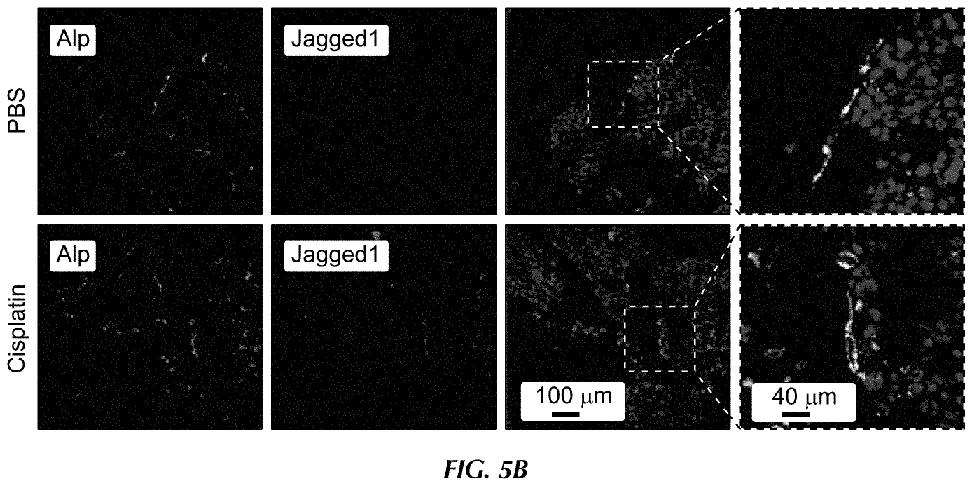

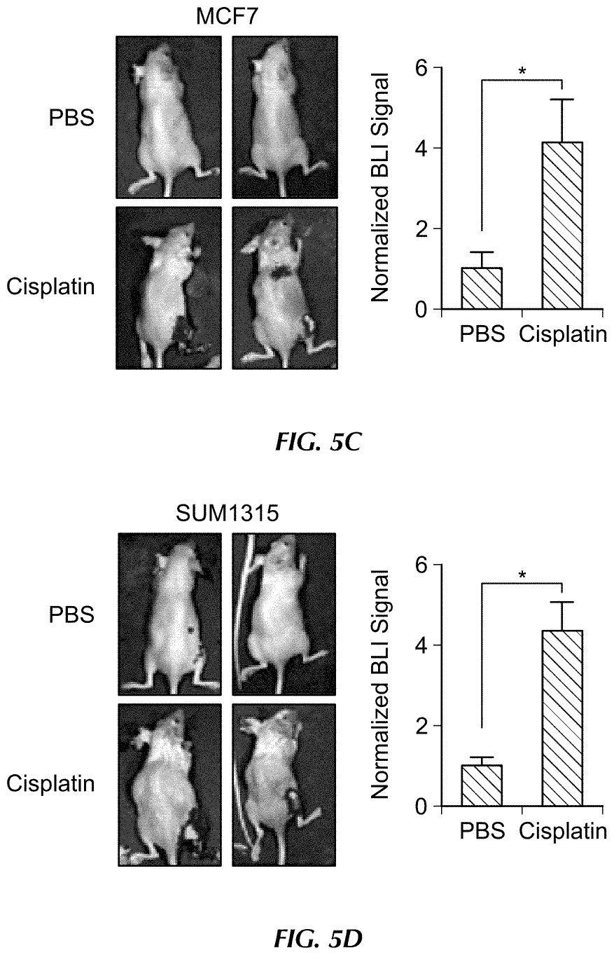

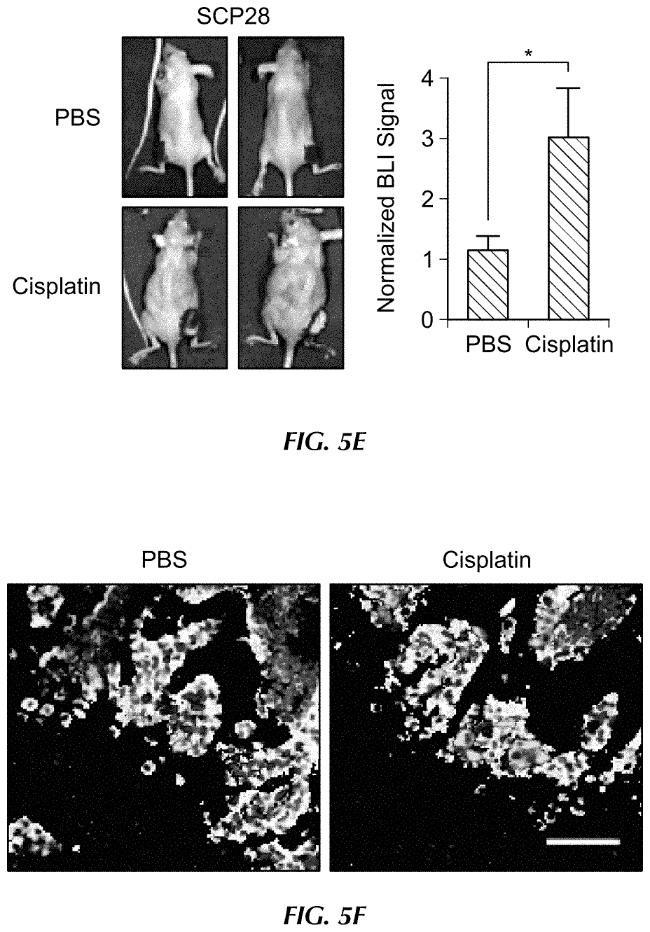

The possibility that chemotherapy agents may induce Jagged1 expression in either tumor cells or in the bone stromal cells was considered. Such chemotherapy-induced Jagged1 might contribute to the resistance of bone metastasis to chemotherapy, and can be targeted by 15D11, as was seen in the combined treatment. Key stromal cell types in bone metastasis include osteoclasts, osteoblasts and their progenitors (such as mesenchymal stem cells (MSCs)), and endothelial cells. Whether Jagged1 could be induced in these cells and various breast cancer cell lines upon treatment of two different chemotherapy agents, paclitaxel and cisplatin, which are commonly used in the treatment of breast cancer, was tested. It was found that Jagged1 expression was significantly increased only in MC3T3-E1 pre-osteoblast cells and MSCs (Ren et al., 2008) (FIG. 5A). There was no significant induction of Jagged1 in endothelial cells, RAW 264.7 pre-osteoclasts, or in SCP28 and SUM1315-M1B1 tumor cells (FIG. 5A).

To confirm this finding in vivo, female nude mice were treated with either PBS or cisplatin. Hind limb bones were dissected 48 hours later and immuno-stained with antibodies against Jagged1 and alkaline phosphatase (Alp), a marker for osteoblast cells. In the PBS control group, Jagged1-positive cells were rarely detected, and mostly co-localizing with ALP-positive osteoblasts. Cisplatin treatment induced much stronger Jagged1 expression based on immunostaining analysis, and these Jagged1-expressing cells again mostly overlapped with Alp positive cells (FIG. 5B). This result thus indicates that chemotherapy induces Jagged1 expression in osteoblast cells in vivo.

### Example 9

### Oxidative Stress Induces Jagged1 Expression in Osteoblastic Lineage Cells

Next, which signaling pathway is responsible for Jagged1 induction after chemotherapy in osteoblast lineage cells was investigated. Chemotherapy generates many stress responses in cells, including endoplasmic reticulum stress (ER stress), and oxidative stress. Some of the stress conditions have been associated with the regulation of Jagged1 expression (Paul et al., 2014). Several treatments were used to mimic or inhibit these stress responses and then Jagged1 mRNA and protein levels were analyzed within the cells. Among these treatments, it was determined that ascorbate, which induces oxidative stress when used at high concentration (Beck et al., 2011), and H₂O₂ induced a strong Jagged1 expression in MSCs, while ER stress inducers Brefeldin A (BFA) and Tunicamycin did not induce Jagged1 expression. Consistent with this observation, cisplatin and docetaxel induced ROS production in MSCs, which could be completely blocked by an ROS inhibitor, N-Acetyl Cysteine. Administration of N-Acetyl Cysteine also completely blocked cisplatin-induced Jagged1 expression in MSCs. These results indicate that Jagged1 is induced in osteoblastic lineage cells during chemotherapy, likely through the ROS pathway.

### Example 10

### Osteoblast-Derived Jagged1 Promotes Bone Metastatic Seeding and Chemoresistance

To directly test the effect of increased Jagged1 expression in osteoblasts on bone metastasis seeding and progression, two models were utilized. In the first model, athymic nude mice were either treated with PBS or cisplatin, which has a much shorter half-life than paclitaxel and facilitated the experimental design. One day after cisplatin treatment, tumor cells were inoculated specifically to the bones in the hind limbs using the intra-iliac artery injection (IIA) method (Wang et al., 2015) to specifically study the stromal effect on bone metastasis seeding after chemotherapy, as tumor cells were not exposed to a clinical dose of cisplatin in this experimental setting. Bone metastatic seeding was tracked by BLI imaging 4 days after tumor cell injection. In all three different cancer cell lines tested (MCF7, SCP28 and SUM1315-M1B1), there was a significant increase of bone metastatic seeding (FIG. 5C-E). Immunostaining confirmed that there were significantly more GFP-positive tumor cells in the bone among mice pre-treated with cisplatin, and these tumor cells were usually located in close proximity to Alp-positive osteoblasts (FIG. 5F).

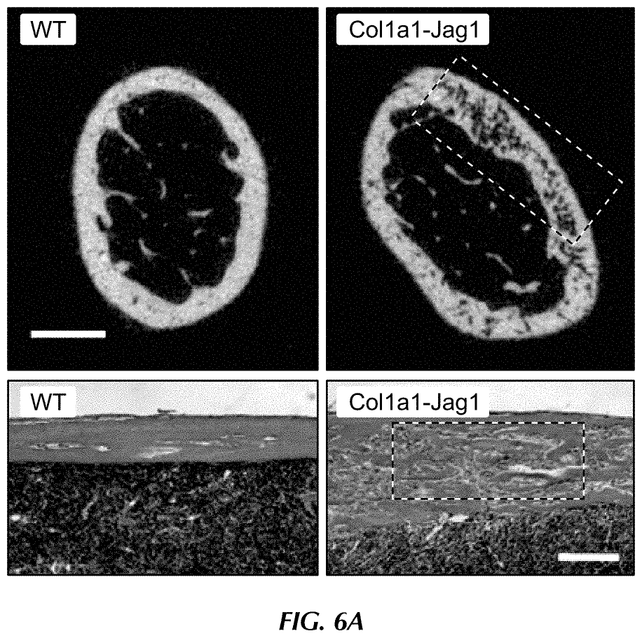

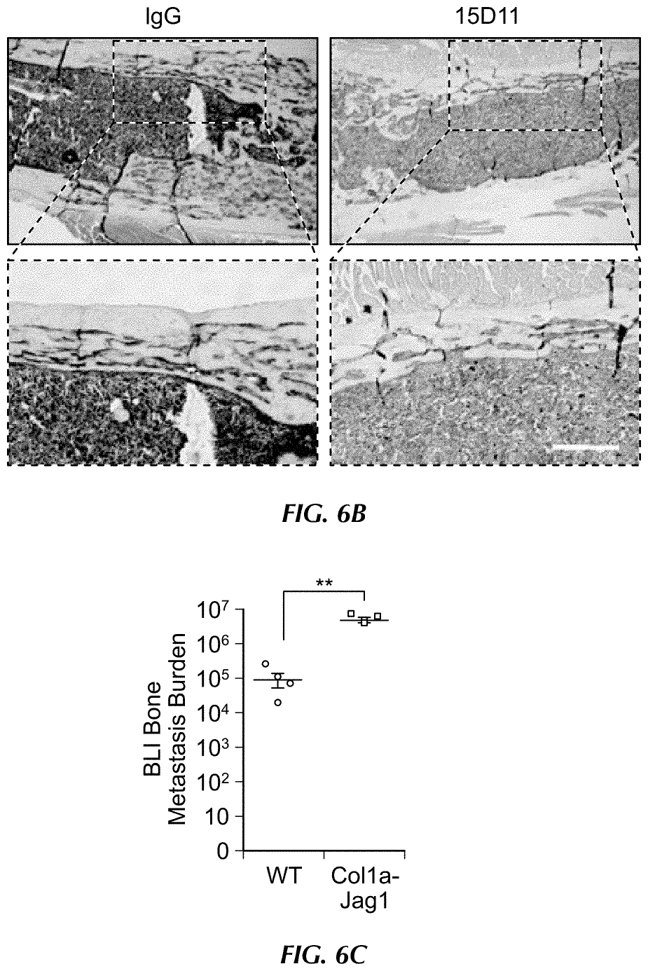

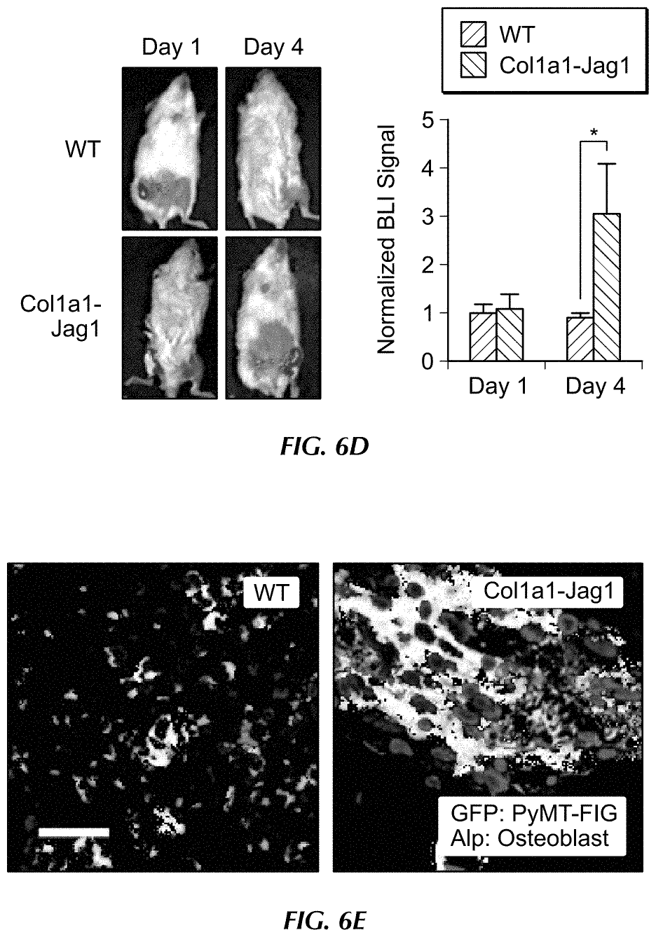

Since chemotherapy may have broad systemic effects on mice that may directly or indirectly influence the outcome of the experiment described above, it was decided to use an alternative approach to specifically increase Jagged1 expression in osteoblasts without using chemotherapy. To this end, a transgenic mouse strain with osteoblast-specific Jagged1 overexpression driven by the Col1a1 promoter was generated. The Col1a1-Jagged1 mice are generally healthy and fertile, but developed a significant shortening and swelling of bone in the middle region of both femur and tibia. μCT imaging and bone histology analysis showed decreased bone density and highly trabecularized bone in the cortical bone area, there was also significant decrease of bone density in trabecular bone region (FIG. 6A). Excessive TRAP-positive osteoclasts were also found in the cortical bone of Col1a1-Jagged1 (FIG. 6B), consistent with the previously reported function of Jagged1 in promoting osteoclastogenesis (Sethi et al., 2011). To test whether these phenotypes were caused by Jagged1 overexpression in osteoblasts, Col1a1-Jagged1 mice were treated with 15D11 for three months. Histological analysis of bone sections indicated that excessive TRAP-positive osteoclasts in Col1a1-Jagged1 mice were reduced significantly after 15D11 treatment, confirming that the phenotype in this mouse strain is indeed caused by osteoblast-derived Jagged1 (FIG. 6B).

Next, long-term bone metastasis progression and short-term metastatic seeding on these mice were tested. For the long-term bone metastasis assay, the syngeneic PyMT-A-FIG cell line previously established in the inventors' lab from a MMTV-PyMT mammary tumor (Wan et al., 2014) and stably labeled with the Firefly-IRES-GFP (FIG) reporter to facilitate in vivo tracking was used. PyMT-A-FIG cells were inoculated into wild-type (WT) or Col1a1-Jagged1 mice via intra-tibial (IT) injection to generate bone metastasis. BLI analysis showed a nearly 20-fold increase of bone metastasis tumor burden in Col1a1-Jagged1 mice than that in WT mice (FIG. 6C). To test short-term bone metastasis seeding, PyMT-A-FIG cells were injected by IIA into either Col1a1-Jagged1 mice or WT mice. Four days after tumor inoculation, there were already ˜3 times more tumor cells surviving in the bone of Col1a1-Jagged1 mice than in WT mice (FIG. 6D). Immunostaining revealed more seeding of GFP-positive tumor cells in bone of Col1a1-Jagged1 mice, often in close contact with ALP-positive osteoblasts (FIG. 6E). Taken together, these results confirmed that osteoblast-derived Jagged1 promotes bone metastatic seeding and outgrowth.

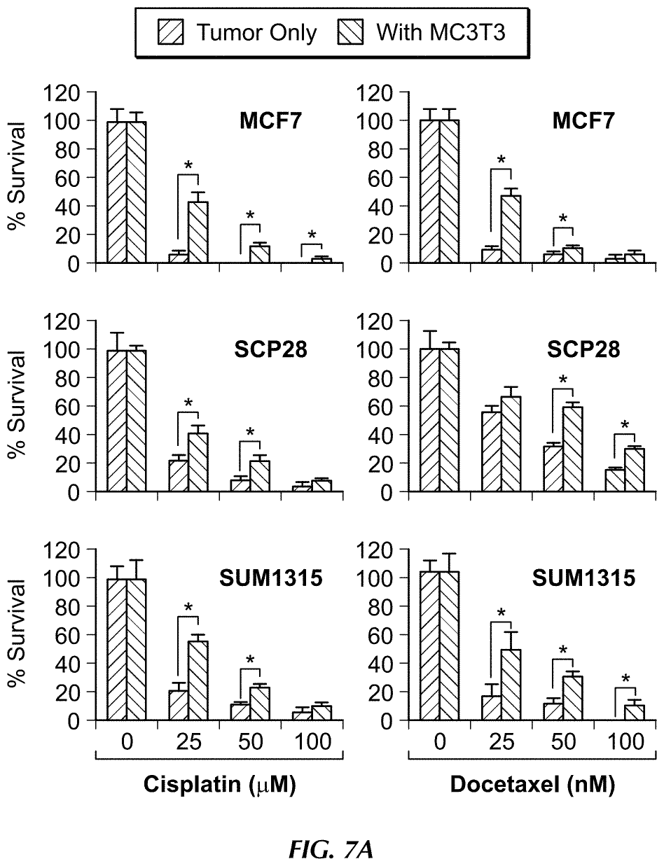

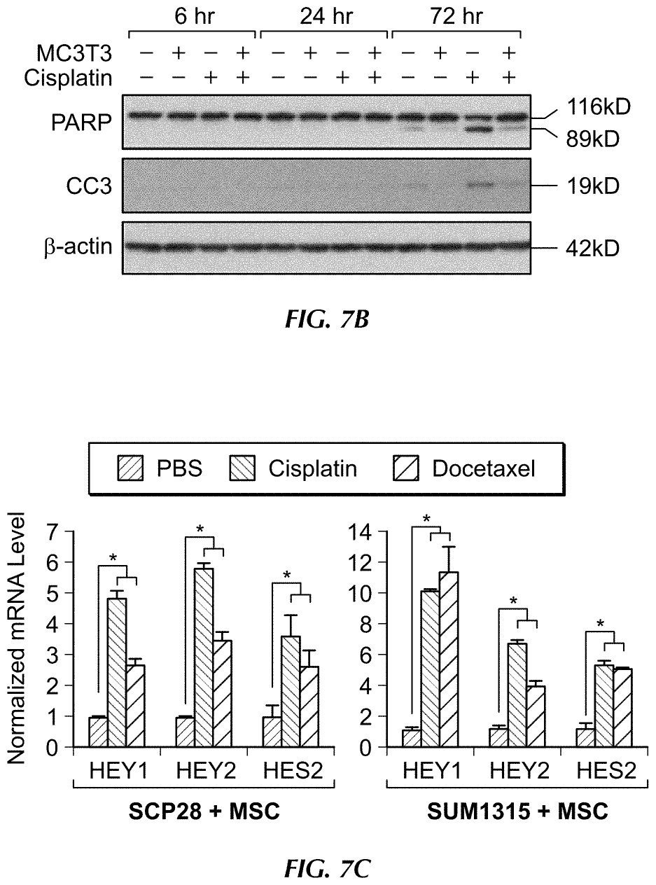

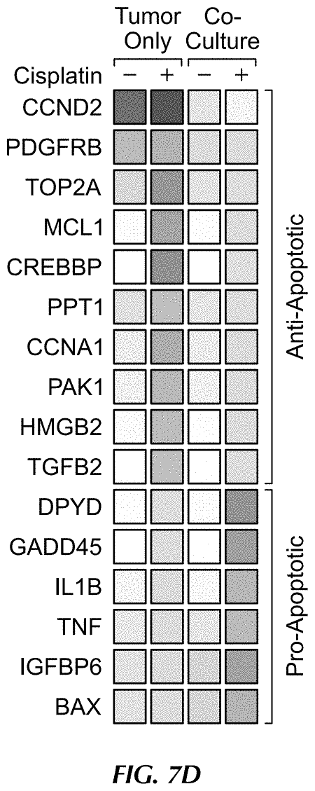

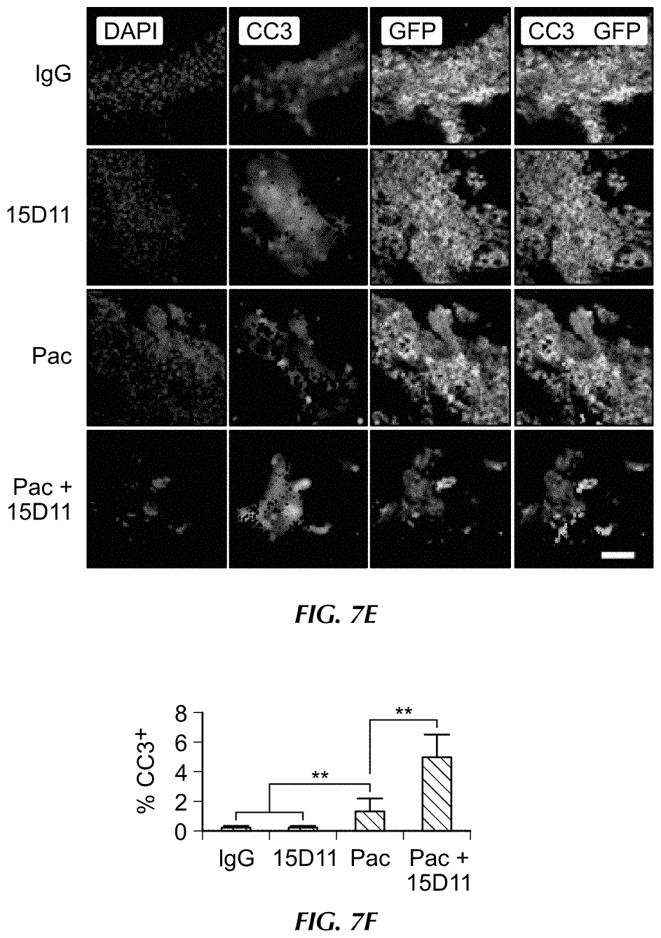

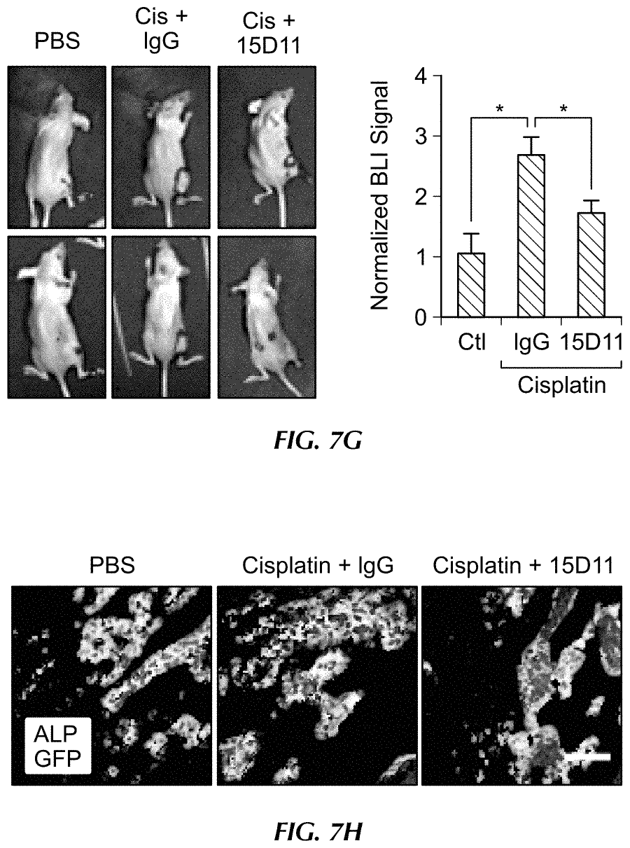

To mimic chemoresistance and tumor seeding effects by osteoblast lineage cells in vitro, an in vitro 3D tumor-stroma co-culture assay (Wang et al., 2015) was adapted. In this assay, tumor cells alone or tumor cells with osteoblast lineage cells were co-cultured in low attachment plates to generate tumor spheres. Consistent with the previous report, when tumor cells were co-cultured with these osteogenic cells, they generated heterotypic spheres, with tumor cells forming an outside shell-like structure and osteogenic cells forming an inner core sphere. There was an increased survival of co-cultured tumor spheres than tumor-only spheres when treated with cisplatin or docetaxel (a potent form of paclitaxel) (FIG. 7A), suggesting that osteoblast lineage cells promote the survival of tumor cells during chemotherapy. To examine cellular apoptotic pathway status, cell lysates were collected from either from SCP28 culture alone or in co-culture with MC3T3 cells. Cleaved PARP and cleaved caspase-3 (CC3) status were analyzed by immuno-blotting at different time points after cisplatin treatment. Seventy-two hours post-treatment of cisplatin, there was a strong induction of cleaved PARP and CC3 in SCP28 singe-cultured cells, while the level of these apoptotic pathway-related proteins was still at low levels in co-culture samples (FIG. 7B). Several studies suggested that osteoblasts and MSCs can secrete fatty acids to promote chemoresistance, and such effect may not require a direct tumor-stroma interaction (Roodhart et al., 2011). To determine whether direct cell-cell contact is needed for the observed chemoresistance, conditional media (CM) was collected from cisplatin-treated MSCs and the same assay was performed. It was found that tumor cells were only partially protected from chemotherapy in the presence of CM compared to higher resistance to chemotherapy of tumor cells co-cultured with MSCs. This result indicates that the direct tumor-stromal cell contact, which is required for Notch signaling, is needed for optimal Jagged1-mediated chemoresistance. Indeed, when Notch downstream gene expression was analyzed in co-cultured tumor cells after either cisplatin or docetaxel treatment, it was found that multiple Notch downstream genes, including human HES2, HEY1, and HEY2, were significantly upregulated in co-culture tumors cells after chemotherapy (FIG. 7C). Co-culture of tumor cells with endothelial cells provided no protection effect during cisplatin or docetaxel treatment, suggesting that only osteoblast lineage cells, but not endothelial cells, protect tumor cells against chemotherapy in the bone.

Notch activation is reported to circumvent apoptosis through affecting the p53-regulated apoptotic pathway (Dotto, 2009; Nair et al., 2003; Wang et al., 2006). The gene expression profile of SUM1315-M1B1 tumor cells before and after cisplatin treatment, and in the presence or absence of osteoblast co-culture, was compared. The analysis focused on 161 apoptosis-related genes. Among apoptosis-related genes that have more than 2 folds differential expression in co-culture versus tumor culture alone in cisplatin treatment conditions, 10 are anti-apoptotic genes that were reduced in expression upon cisplatin treatment in tumor culture alone, but were elevated in basal expression levels and further increased upon cisplatin treatment in the co-culture condition. Similarly, 6 pro-apoptotic genes were induced in tumor cells alone by cisplatin treatment, but such change was suppressed in the co-culture condition (FIG. 7D). Notably, among those 16 genes, at least 5 genes are known to be regulated by the p53 pathway. MCL1, CCNA1, GADD45, and BAX are direct transcriptional targets of p53 and CCND2 is an indirect p53 pathway gene. Taken together, these results suggested that osteoblast-mediated chemoresistance of tumor cells is at least in part mediated by Notch activation and its effect on suppressing p53-regulated apoptotic pathway.

To directly analyze whether Jagged1 protects tumor cells from chemotherapy-induced apoptosis, immunofluorescent staining of GFP-positive tumor cells and CC3 of mouse bone sections after two rounds of single or combined treatments of paclitaxel and 15D11, as in the experiment in FIG. 4A, were performed. In either the IgG or 15D11 treated group, there was almost no detectable CC3 staining. While substantial numbers of tumor cells in the paclitaxel treatment group were positive for CC3, the most abundant CC3 positive tumor cells were observed in paclitaxel and 15D11 combinational treatment group (FIGS. 7E and 7F).

### Example 11

### Combinatorial Treatment of 15D11 and Chemotherapy Synergistically Reduces the Incidence of Spontaneous Bone Metastasis

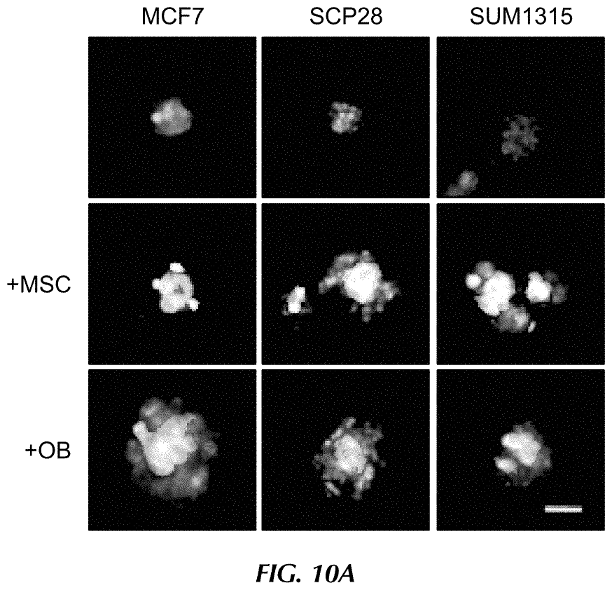

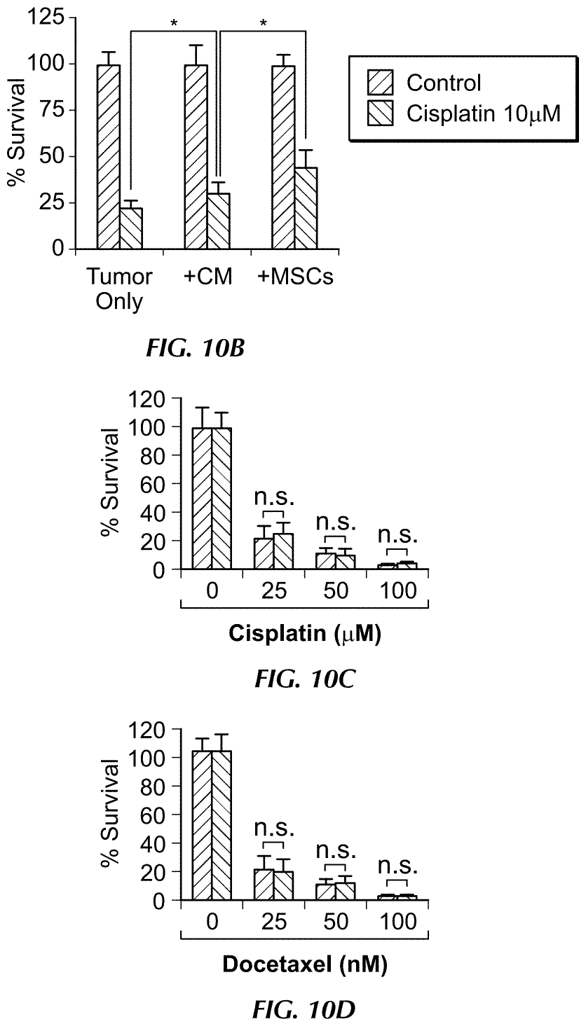

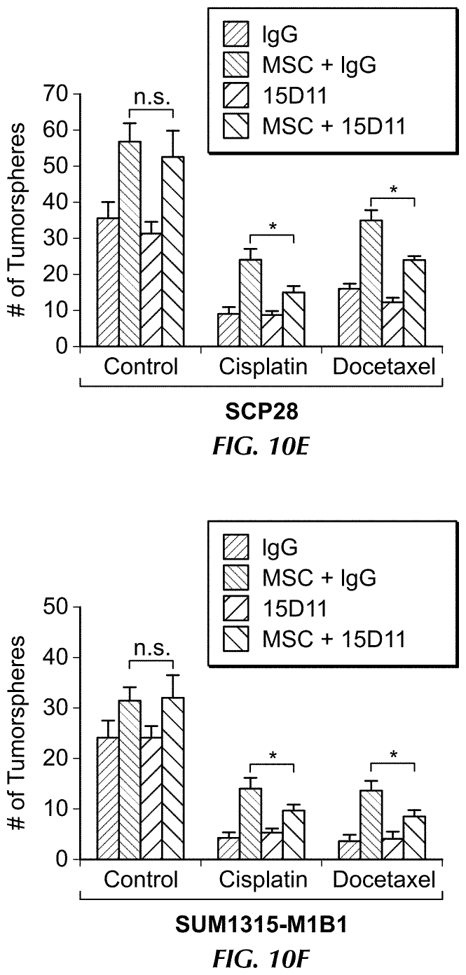

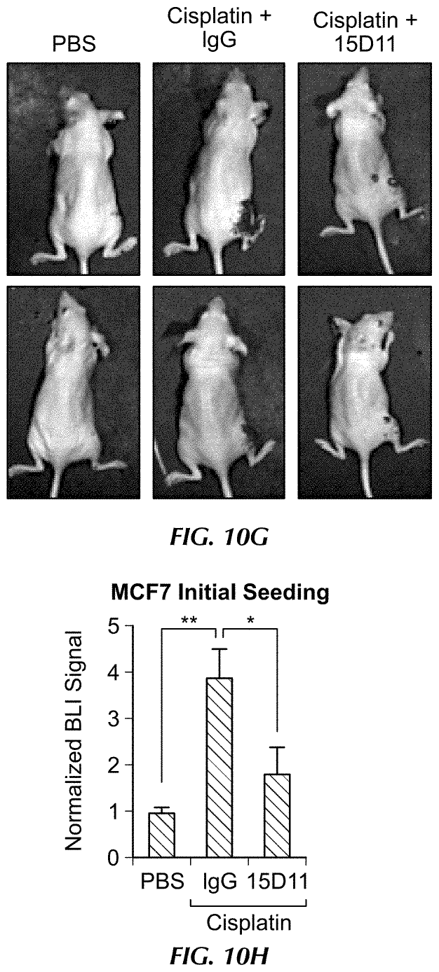

To test whether anti-Jagged1 antibody 15D11 could block the tumor cell survival effects of chemotherapy, SCP28 and SUM1315-M1B1 cells were co-cultured with MSCs. At the time of treating cells with chemotherapy, cells were also incubated with IgG or 15D11 antibody. Administration of 15D11 significantly reversed the increase of the number of surviving tumor spheres when the tumor cells were co-cultured with MSCs (FIGS. 10E and 10F). Similar findings were observed in co-culture experiment with MC3T3 cells. To confirm this result in vivo, we treated nude mice with cisplatin one day before the tumor cell injection. Right at the time of IIA injection of SUM1315-M1B1 tumor cell, mice were also treated with either IgG or 15D11 antibody. Bone metastatic seeding was tracked by BLI and by IF staining 4 days after the injection. Similar to the earlier experiment (FIGS. 5C-E), more tumor cells were seeded in the bone of the mice pre-treated with cisplatin (FIG. 7G). Such an increase of metastatic seeding in chemotherapy-primed bone was largely eliminated by 15D11 treatment. Similar result was also obtained using ER+ MCF7 breast cancer cells (FIG. 10G-H). Confocal IF imaging of bone sections from euthanized animals further confirmed increased seeding of tumor cells next to ALP-positive osteoblasts in chemotherapy-primed mice, and the subsequent decrease after treatment with 15D11 (FIG. 7H). In conclusion, these results demonstrate that although chemotherapy is cytotoxic to tumor cells, its full therapeutic potential is compromised by the increased expression of Jagged1 in osteoblast lineage cells in response to chemotherapy. Osteoblast derived-Jagged1 activates Notch signaling in tumor cells to promote resistance to chemotherapy-induced apoptosis. Combining chemotherapy with 15D11 treatment nullifies such stroma-mediated mechanism of chemoresistance, and achieve optimal outcome in the treatment of bone metastasis.

The mouse model study revealed a role of chemotherapy-induced Jagged1 in osteoblasts in promoting chemoresistance of bone metastasis. To confirm whether Jagged1 is also induced in osteoblasts in human cancer patients receiving chemotherapy, paired bone marrow cytospin samples of patients before and after receiving the adjuvant chemotherapy of carboplatin and paclitaxel were obtained and immunostaining analysis was performed on the samples. Prior to chemotherapy, Jagged1 was expressed at a low level, mostly on ALP-positive osteoblasts (FIG. 8A). After chemotherapy, there was a significant increase of Jagged1 staining on osteoblasts (FIGS. 8A and 8B), consistent with our observation from in vitro and in mouse models (FIGS. 5A and 5B).

In the clinical management of early stage breast cancer, neoadjuvant and adjuvant chemotherapy is commonly used to prevent future relapse. Although chemotherapy certainly decreases local relapse and bone metastasis, its effect is potentially diminished by the chemotherapy-induced osteoblast Jagged1 in promoting metastatic seeding in bone. Therefore, combining chemotherapy and 15D11 treatment may further decrease the risk of bone relapse. To test this notion, 4T1.2 mammary tumor cells, which can spontaneously metastasize to the bone from the mammary glands (Eckhardt et al., 2005), were utilized. 4T1.2 tumor cells were injected into mammary fat pads of female Balb/c mice to allow the establishment of primary tumors. Once the primary tumor sizes reached 5 mm, mice were randomly grouped for treatment with IgG, paclitaxel, 15D11, or both paclitaxel and 15D11 (FIG. 8C). Based on X-ray images, it was noticed that over 50% of mice in IgG control group developed bone metastasis in their hind limb bones. 15D11 or paclitaxel treatment reduced bone metastasis incidence by 15-30%, while a strong inhibition of bone metastasis was achieved by paclitaxel and 15D11 combinatorial treatment, with only 10% of mice developed bone metastasis (FIGS. 8D and 8E). Bone lesion areas were also significantly reduced in combinational treatment (FIG. 8F). Overall, these results suggest that treating cancer patients with adjuvant chemotherapy combined with an anti-Jagged1 antibody such as 15D11 could potentially reduce the incidence of future bone metastasis relapse.

### Example 12

### Materials and Methods

Animal Models

Mice were purchased from Jackson laboratory. For orthotopic primary tumor formation and spontaneous bone metastasis assay, female BALB/c mice (4-6 weeks old) were anaesthetized and a small incision was made to reveal the mammary gland. 10⁵ GFP labeled 4T1.2 tumor cells suspended in 10 μl PBS were injected directly into the mammary fat pad. Spontaneous bone metastasis was monitored by X-ray imaging. Mice were euthanized when the primary tumor reaches 20×20 mm. For experimental bone metastasis, three models were used using intra-cardiac (IC), intra-iliac artery (IIA), and intra-tibia (i.t.) injection methods, as previously described (Andersen et al., 2003; Sethi et al., 2011; Wang et al., 2015). Bone metastasis burden was monitored by weekly BLI and X-ray imaging. At the experimental endpoint, mice were euthanized for bone histology analysis and half of the bone samples were used for μCT imaging and bone density quantification.

Clinical Bone Marrow Cytospin Analysis

14 pairs of bone marrow cytospin samples (without any DTCs) from cancer patients before and after carboplatinum and paclitaxel were acquired from the University Hospital Essen, Essen, Germany. Cytospin slides were dried on room temperature for 1 hour before staining. Slides were thawed for 3 min and fixed for 10 min with 4% PFA in PBS before permeabilized in 0.2% Triton X-100 in PBS for 10 minutes and blocked in 10% goat serum for 20 minutes. Samples were then incubated with Jagged1 primary antibody for 1 hour and in secondary antibody for 45 minutes. Samples were blocked for 20 minutes before incubation with the next set of primary (Alkinine Phosphatase antibody) and secondary antibodies. All antibodies were diluted in 10% goat serum. Slides were stained with Hoechst and mounted with coverslips using Prolong gold antifade reagent.

Cell Lines

293T, H29-Clone #7, MC3T3-E1 Clone #4(denoted as MC3T3 for abbreviation thereafter), MCF7TR, mouse mesenchymal stromal cell (MSC, isolated from BALB/c), 4T1.2, SCP28, SCP28-Vector and -Jagged1 cells, SCP28, RAW264.7, and SUM1315-M1B1 cells were cultured according to American Type Culture Collection instructions and the inventors' previous studies (Ell et al., 2013; Ren et al., 2008; Sethi et al., 2011; Zheng et al., 2014). MSC cells were derived previously and were cultured in DMEM supplemented with 10% FBS, 30 ng/ml bFGF, 100 U/ml penicillin, and 100 μg/ml streptomycin (Ren et al., 2008). All plasmids were transfected into different cell lines using Lipofectamine 2000 following the manufacturer's manual (Life Technologies, CA, USA). To generate stable cell lines, a pLEX-MCS based lentivirus vector or a pMSCV-hygro based retrovirus vector was used in our study. Lentiviruses were packaged in HEK293T cells while retroviruses were packaged by using H29 cell line. Conditional media from these packaging cells containing viruses were collected 2 days and 3 days after transfection. Recipient cell lines were exposed to conditional media containing viruses supplemented with 2 μg/mL Polybrene for 48 h. Infected cells were selected with puromycin or hygromycin to generate stable expressing cell lines. 293T and H29clone #7 are cell lines from female humans. All other cancer cell lines are breast cancer cell lines from either female human or mouse.

Osteoclast Differentiation Assay

For osteoclast differentiation assays, RAW264.7 cells were seeded onto Fc- or recombinant Jagged1 protein plates, or co-cultured with indicated cell lines at the concentration of 0.2 million cells/well in 12 well plate. Cells were then treated with 5 ng/ml RANKL in DMEM plus 10% FBS, with media changed every 2 days and twice a day from Day 5 and on. Primary pre-osteoclasts were isolated from bone marrow cells flushed from the tibia of 6-week-old WT Balb/c mice and filtered through a 70 μm cell-strainer before overnight culture in α-MEM with 10% FBS. The following day non-adherent cells were plated and supplemented with 50 ng/ml M-CSF for 2 days. Cells were then re-plated onto Fc- or recombinant Jagged1 protein plates, or co-cultured with indicated cell lines in 12 well plate at the presence of 5 ng/ml RANKL in DMEM plus 10% FBS, with media changed every 2 days (media was changed twice a day from Day 5 and on). Mature osteoclasts were TRAP stained using a leukocyte acid phosphatase kit (387A-1KT Sigma) and TRAP positive and multinucleated cells were quantified as mature osteoclasts.

2-D and 3-D Tumor-Stromal Co-Culture

For 2-D tumor osteoblast co-culture, SCP28-vector or SCP28-Jagged1 tumor cells were cultured with MC3T3 cells in 10 cm plate in DMEM with 10% FBS. One day after co-culture, media was changed to serum free DMEM for another 24 hours before the conditional media collection. The collected conditional media were concentrated by centrifuging at room temperature at 4000 rpm using Amicon Ultra-15 (3K) centrifuge filter (UFC900324, EMD Millipore, MA, USA) for further usage. For tumor-stroma cell 3-D co-culture assay, 1:1 ratio, a total of 10000 tumor cells and MC3T3 cells (or MSCs) were cultured in a serum free mammosphere formation media in low attachment plates (Corning, Corning N.Y., USA). The media is freshly prepared with 1 ml B27 (Life Technologies, CA, USA), 20 ng/ml bFGF (Novoprotein, NJ, USA), 20 ng/ml EGF (Novoprotein, NJ, USA), 100 μg/ml Gentamycin (Life Technologies, CA, USA), and 0.25 ml non-essential amino acid solution (Life Technologies, CA, USA) in a total of 50 ml DMEM/F12 media. Cells were cultured to form 3-D spheres before treating with respective chemotherapeutic agent.

Labeling of MC3T3 and MSC Cells

To generate labeled MSC and MC3T3 cells for co-culture study, these two cell lines were infected with pLEX-mCherry lentivirus for two days and let the cells expand for another 5 days. Cultured cells were then trypsin digested and re-suspended in FACS buffer (PBS supplemented with 5% newborn calf serum) and filtered through 70 mm nylon cell strainers before flow cytometric analysis on a FACS sort instrument (BD Biosciences). Non-labeled parental cells were used as negative control. DAPI was used for nuclear counter staining to eliminate dead cells.

Generation and Characterization of 15D11 Antibody Immunization

Fully human antibodies to Jagged1 were generated using XenoMouse technology, transgenic mice engineered to express diverse repertoires of fully human IgGx and IgGX, antibodies of the corresponding isotypes (Kellermann and Green, 2002; Mendez et al., 1997). XMG2-KL and XMG4-KL strains of mice were immunized with CHO transfectants expressing full length human Jagged1. Cellular immunogens were dosed at 4.0×10⁶ Jagged1 transfected cells/mouse and subsequent boosts were of 2.0×10⁶ Jagged1 transfected cells/mouse. Injection sites used were combinations of subcutaneous base-of-tail and intraperitoneal. Adjuvant used was Alum (E.M. Sergent Pulp and Chemical Co., Clifton, N.J., cat. #1452-250). Mice were immunized over a period of 8 weeks to 12 weeks.

Antibody Specificity Determination

After 14 days of culture, hybridoma supernatants were screened for human Jagged1 specific monoclonal antibodies by Fluorometric Microvolume Assay Technology (FMAT) (Applied Biosystems, Foster City, Calif.). The supernatants were screened against 293T cells transiently transfected with human Jagged1 and counter screened against 293T cells transiently transfected with the same expression plasmid that did not contain the JAGGED1 gene.

Ligand Binding Affinity Test

A ligand binding competition method was developed to identify antibodies (in the hybridoma supernatants) that bind Jagged1 ligand and block binding of three receptors: Notch-3, Notch 2 and Notch-1. FACS assays were performed by incubating 20 μl of hybridoma supernatants with 50000 cells transiently expressing at 4° C. for one hour followed by two washes with PBS/BSA. Cells were then treated with 5 μg/ml fluorochrome-labeled Notch-3 (#1559-NT, R&D Systems) at 4° C. followed by two washes. The cells were re-suspended in 1 ml PBS/BSA and antibody binding was analyzed using a FACS Calibur™ instrument. Similar assays were performed using Notch-2 (#3735-NT, R&D Systems) and Notch-1 (#3647-TK, R&D Systems). The experiments included negative control hybridoma supernatants. The average signal observed in these negative control experiments was adopted as the maximum possible signal for the assay. Experimental supernatants were compared to this maximum signal and a percent inhibition was calculated for each well (% Inhibition=(1−(FL1 of the anti-BCMA hybridoma supernatant/Maximum FL1 signal))).

Additional Binding Characterization

FACS binding assays were performed to evaluate the binding of the anti-Jagged1 specific antibodies to the murine Jagged1 as well as related Notch ligands, human JAGGED2 and human DLL4. FACS assays were performed by incubating hybridoma supernatants with 50000 cells at 4° C. for one hour followed by two washes with PBS/BSA. Cells were then treated with fluorochrome-labeled secondary antibodies at 4° C. followed by two washes. The cells were re-suspended in 1 ml PBS/BSA and antibody binding was analyzed using a FACSCalibur™ instrument.

Kd Estimation by KinExA

Binding of anti-Jagged1 antibody 15D11 with 293T/muJagged1 clonel cells were tested on KinExA. Briefly, UltraLink Biosupport (Pierce cat #53110) was pre-coated with goat-anti-huFc (Jackson Immuno Research cat #109-005-098) and blocked with BSA. 10 pM and 100 pM of Ab 15D11 was incubated with various density (1.5×10²-9.0×10⁶ cell/ml) of 293T cells expressing muJagged1 in 1% FBS, 0.05% sodium azide, DMEM. Samples containing 15D11 antibody and whole cells were rocked for 4 hours at room temperature. The whole cells and antibody-cell complexes were separated from unbound free antibody using Beckman GS-6R centrifuge at approximately 220×g for 5 min. The supernatant was filtered through 0.22 μm-filter before passing the goat-anti-huFc-coated beads. The amount of the bead-bound Ab 15D11 was quantified by fluorescent (Cy5) labeled goat anti-humanIgG (H+L) antibody (Jackson Immuno Research cat #109-175-088). The binding signal is proportional to the concentration of free Ab 15D11 in solution at each cell density. Equilibrium dissociation constant (Kd) was estimated using model of unknown ligand for n-curve analysis in KinExA™ Pro software.

In Vivo Treatment Schedule and Dosing

The initial treatment is either one day before tumor cell injection or one week after tumor cell injection as described in each experiment. For IgG or 15D11 antibody treatment, mice were i.p. injected at the dosing of 10 mg/kg, twice a week. For OPG-Fc treatment, mice were i.p. injected at the dosing of 3 mg/kg, twice a week. For paclitaxel treatment, paclitaxol powder was first dissolved in 95% ethanol, sonicated and shaken overnight, before mixing 1:1 with cremophor (Sigma Catalog#:769193-1KG) to reach the final concentration of 20 mg/ml. Paclitaxel was diluted 5 times in PBS right before usage. Mice were i.v. injected at 25 mg/kg for the first time, and then 20 mg/kg each time, twice a week for up to total of 6 times. Cisplatin was used at the dosing of 2 mg/kg in experiment in FIG. 5D. GSI inhibitor (MRK003) was prepared and used similarly as described before (Sethi et al., 2011).

Angiogenesis Effect of 15D11 Antibody

The angiogenesis effect of 15D11 antibody is determined by two methods: 1) Mouse neonatal retina study. Mouse retinas were harvested, stained and fixed as described similarly in a previous study (Ridgway et al., 2006); and 2) Tumor angiogenesis model. 2×10⁶ Colo 205 cells were implanted into female athymic nude mice subcutaneously. Mice were randomized at Day 11 before treated with IgG or 15D11 antibody at the dosage of 300 μg/mouse for twice a week. Tumor volume was monitored by palpation. To determine tumor angiogenesis effect, Colo205 tumors from mice treated for 96 hours with 500 μg of IgG, or 15D11 antibody were collected. Intra-tumor blood vessels were visualized by CD31 staining and counterstained with hematoxylin. (n=3 per group). No vascular differences between groups were observed.

Bone Histology Analysis

Hindlimb bones were excised from mice at the end point of each experiment, immediately after the last BLI time point. Following this, the tumor-bearing hind limb bones were fixed in 10% neutral-buffered formalin, decalcified in 10% EDTA for 2 weeks, and embedded in paraffin for hematoxylin and eosin (H&E), tartrate-resistant acid phosphatase (TRAP) (Kos et al., 2003), or immunohistochemical staining. Histomorphometric analysis was performed on H&E stained bone metastasis samples using the Zeiss Axiovert 200 microscope and the AxioVision software version 4.6.3 SP1. For quantitative analysis of lesion area, a 10× objective was used to focus on the tumor region of interest and images were acquired using the AxioCamICc3 camera set to an exposure of around 100 ms. Osteoclast number was assessed as multinucleated TRAP+ cells and reported as number/field. For immunofluorescence staining of the bone samples, Hind limb bones were excised from mice at the end point of each experiment immediately followed by PBS and 4% freshly prepared paraformaldehyde. Non-decalcified bone samples were then frozen in embedding media and sectioned with Leica CM3050S Research Cryostat at 20 μM with Cryofilm type IIIC (Section-Lab, Japan) and stained with respective primary antibodies and fluorophore labeled secondary antibodies. GFP antibody: Cat #ab13970 (Abcam). Jagged1 antibody: Cat #AP09127PU-N (Acris Antibodies). ALP antibody: Cat #MAB29091 (R&D Systems). Cleaved Caspase-3 antibody: Cat #9661S (Cell Signaling). Ki67 antibody: Cat #ab15580 (Abcam). Images were taken using Nikon A1 confocal microscope at Princeton University Molecular Biology Confocal microscope core facility.

μCT Analysis

Femurs and tibias were scanned using the INVEON PET/CT (Siemens Healthcare) at the Preclinical Imaging Shared Resource of Cancer Institute of New Jersey. The X-Ray tube settings were 80 kV and 500 μA and images were acquired at the highest resolution without CCD binning, resulting in a voxel size of 9.44 μm. A 0.66° rotation step through a 195° angular range with 6500 ms exposure was used. The images were reconstructed with Beam Hardening Correction and Hounsfield calibration before being analyzed using the INVEON Research Workplace software (Siemens Healthcare). After processing with a 3-D Gaussian filter to reduce noise, ROI's were manually segmented that corresponded to the cortical and trabecular bone regions.

X-Ray Imaging and Osteolytic Lesion Quantification

Osteolytic bone lesions in mice were assessed by X-ray radiography. Anesthetized mice were placed on individually wrapped films (BIOMAX XAR Film, Cat#: F5763-50EA, Sigma-Aldrich) and exposed to X-ray radiography at 35 kV for 15 seconds using a MX-20 Faxitron instrument. Films were developed using a Konica SRX-101A processor. Changes in bone remodeling and osteolytic lesions (radiolucent lesions) in the hind limbs of mice were identified and quantified using the Adobe Photoshop software (Adobe Systems Inc.)

Molecular Cloning and Transgenic Mouse Generation

The Jagged1 overexpression plasmids were generated as described in previously published results (Sethi et al., 2011). To generate the Col1a1-mJagged1 mouse strain, a backbone vector of pTyr-Co1a1 was used. Mouse cDNA library was generated using a mouse mammary gland mRNA from FVB background mice and reverse transcribed to cDNA by using SuperScript III First Strand Synthesis System for RT-PCR (118080-051 Life Technologies CA). mJagged1 cDNA was PCR amplified using this library by a pair of primers both with SwaI restriction enzyme overhangs (CATTATTTAAATgccaccatgcggtccccacgga (SEQ. ID. NO: 65) and CATT ATTTAAATctgctatacgatgtattccatccgg (SEQ. ID. NO: 66)) and inserted into SwaI site of pTyr-Col1a1 plasmid with sequencing confirmation. The genotyping was performed using primer pairs of CAACACCACGGAATTGTCAGT (SEQ. ID. NO: 67) and GATGATGGGAACCCTGTCAA (SEQ. ID. NO: 68) with final PCR product of 1012 bps.

Western Blot Analyses

SDS lysis buffer (0.05 mM Tris-HCl, 50 mM BME, 2% SDS, 0.1% Bromophenol blue, 10% glycerol) was used to collect protein from cells. Samples were heat denatured and equally loaded, separated on a 10% SDS-page gel, transferred onto a PVDF membrane (Millipore), and blocked with 5% milk. Primary antibodies for immunoblotting included: anti-β-actin (1:10,000 dilution, Abcam, cat #ab6276, clone AC-15), anti-Cleaved Caspase 3 (1:1000 dilution, Cell Signaling, Cat #9661S), anti-IL-6 (1:1000 dilution, MLB International Cooperation, cat #JM-5144-100), anti-PARP (1:1000 dilution, Cell Signaling, Cat #95325), anti-Jagged1 (1:1,000 dilution, Santa Cruz Biotechnology, Cat #SC8303). Membranes were incubated with horseradish peroxidase (HRP)-conjugated anti-mouse, rabbit or rat secondary antibody (1:2000 dilution, GE Healthcare) for 1 h and chemiluminescence signals were detected by ECL substrate (GE Healthcare).

RNA Isolation and qRT-PCR Analysis

Total RNAs were isolated from cells using RNeasy kit (Qiagen) following manufacturer's instructions. RNAs were reverse transcribed into cDNAs by using Superscript III reverse transcription kit (Invitrogen). Real-time RT-PCR was performed on ABI 7900 96 HT series PCR machine (Applied Biosystem) using SYBR Green Supermix (Bio-Rad Laboratories). The gene-specific primer sets were used at a final concentration of 0.5 μM. All real-time RT-PCR assays were performed in duplicate in at least two independent experiments. Relative expression values of each target gene were normalized to GAPDH mRNA level.

Microarray and Heatmap Generation

SUM1315-M1B1 breast cancer cells (GFP labeled) were cultured alone or co-cultured with MC3T3-E1 clone #4 bone osteoblast cells, and treated with control PBS or 10 μM cisplatin for 48 hr. Tumor cells were sorted using FACS based on GFP labeling. RNA was collected from these samples using the RNAeasy Mini Kit (Qiagen, Valencia Va.) according to manufacturer's instructions. The gene expression profiles were determined using Agilent whole human genome microarray 4×44K G4112F, following the manufacturer's instructions. Briefly, the RNA samples and universal human reference RNA (Agilent 740000) were labeled with CTP-cy5 and CTP-cy3, respectively, using the Agilent Quick Amp Labeling Kit. Labeled testing and reference RNA samples were mixed in equal proportions, and hybridized to the human GE 4×44K array. The arrays were scanned with an Agilent G2505C scanner and raw data was extracted using Agilent Feature Extraction software (v11.0). Data was analyzed using the GeneSpring 13 software (Agilent). The expression value of individual probes refers to the Log 2(Cy5/Cy3) ratio. The microarray dataset is deposited in GEO database with accession number GSE97997. Heat map representation of microarray data displays the selected pro- and anti-apoptosis genes from Hallmark-apoptosis signature in MsigDB (http://software.broadinstitute.org/gsea/msigdb/cards/HALLMARK_APOPTOSIS.html). The gene expression values of these genes are further transformed based on median of samples from paired culture conditions (alone vs co-culture) in both saline and cisplatin treatments.

Quantification and Statistical Analysis

Results were reported as mean±SD (standard deviation) or mean±SEM (standard error of the mean), as indicated in the figure legend. Statistical comparisons were performed using unpaired two-sided Student's t-test with unequal variance assumption and by Mann-Whitney U test. All in vitro experiments were repeated three times and animal experiments were repeated once. All the experiments with representative images (including western blot and immunofluorescence) have been repeated at least twice and representative images were shown (except for bone marrow samples). The IF images in FIG. 8 are representative of bone marrow cytospin specimens of the same category (pre-treatment vs post-treatment). Quantification of these IF images was performed using ImageJ software.

### REFERENCES

Anampa, J., Makower, D., and Sparano, J. A. (2015). Progress in adjuvant chemotherapy for breast cancer: an overview. BMC Med 13, 195.

Andersen, C., Bagi, C. M., and Adams, S. W. (2003). Intra-tibial injection of human prostate cancer cell line CWR22 elicits osteoblastic response in immunodeficient rats. Journal of musculoskeletal & neuronal interactions 3, 148-155.

Bargman, R., Posham, R., Boskey, A., Carter, E., DiCarlo, E., Verdelis, K., Raggio, C., and Pleshko, N. (2012). High- and low-dose OPG-Fc cause osteopetrosis-like changes in infant mice. Pediatric research 72, 495-501.

Beck, R., Pedrosa, R. C., Dejeans, N., Glorieux, C., Leveque, P., Gallez, B., Taper, H., Eeckhoudt, S., Knoops, L., Calderon, P. B., and Verrax, J. (2011). Ascorbate/menadione-induced oxidative stress kills cancer cells that express normal or mutated forms of the oncogenic protein Bcr-Abl. An in vitro and in vivo mechanistic study. Investigational new drugs 29, 891-900.

Benedito, R., Roca, C., Sorensen, I., Adams, S., Gossler, A., Fruttiger, M., and Adams, R. H. (2009). The notch ligands Dll4 and Jagged1 have opposing effects on angiogenesis. Cell 137, 1124-1135.

Boelens, M. C., Wu, T. J., Nabet, B. Y., Xu, B., Qiu, Y., Yoon, T., Azzam, D. J., Twyman-Saint Victor, C., Wiemann, B. Z., Ishwaran, H., et al. (2014). Exosome transfer from stromal to breast cancer cells regulates therapy resistance pathways. Cell 159, 499-513.

Braun, S., Vogl, F. D., Naume, B., Janni, W., Osborne, M. P., Coombes, R. C., Schlimok, G., Diel, I. J., Gerber, B., Gebauer, G., et al. (2005). A pooled analysis of bone marrow micrometastasis in breast cancer. The New England journal of medicine 353, 793-802.

Cao, Z., Ding, B. S., Guo, P., Lee, S. B., Butler, J. M., Casey, S. C., Simons, M., Tam, W., Felsher, D. W., Shido, K., et al. (2014). Angiocrine factors deployed by tumor vascular niche induce B cell lymphoma invasiveness and chemoresistance. Cancer cell 25, 350-365.

Choy, L., Hagenbeek, T. J., Solon, M., French, D., Finkle, D., Shelton, A., Venook, R., Brauer, M. J., and Siebel, C. W. (2017). Constitutive NOTCH3 Signaling Promotes the Growth of Basal Breast Cancers. Cancer Res 77, 1439-1452.

Coleman, R. E., Guise, T. A., Lipton, A., Roodman, G. D., Berenson, J. R., Body, J. J., Boyce, B. F., Calvi, L. M., Hadji, P., McCloskey, E. V., et al. (2008). Advancing treatment for metastatic bone cancer: consensus recommendations from the Second Cambridge Conference. Clinical cancer research : an official journal of the American Association for Cancer Research 14, 6387-6395.

Dotto, G. P. (2009). Crosstalk of Notch with p53 and p63 in cancer growth control. Nat Rev Cancer 9, 587-595.

Duan, C. W., Shi, J., Chen, J., Wang, B., Yu, Y. H., Qin, X., Zhou, X. C., Cai, Y. J., Li, Z. Q., Zhang, F., et al. (2014). Leukemia propagating cells rebuild an evolving niche in response to therapy. Cancer cell 25, 778-793.

Eckhardt, B. L., Parker, B. S., van Laar, R. K., Restall, C. M., Natoli, A. L., Tavaria, M. D., Stanley, K. L., Sloan, E. K., Moseley, J. M., and Anderson, R. L. (2005). Genomic analysis of a spontaneous model of breast cancer metastasis to bone reveals a role for the extracellular matrix. Molecular cancer research: MCR 3, 1-13.

Ell, B., Mercatali, L., Ibrahim, T., Campbell, N., Schwarzenbach, H., Pantel, K., Amadori, D., and Kang, Y. (2013). Tumor-induced osteoclast miRNA changes as regulators and biomarkers of osteolytic bone metastasis. Cancer cell 24, 542-556.

Forozan, F., Veldman, R., Ammerman, C. A., Parsa, N. Z., Kallioniemi, A., Kallioniemi, O. P., and Ethier, S. P. (1999). Molecular cytogenetic analysis of 11 new breast cancer cell lines. Br J Cancer 81, 1328-1334.

Gu, B., Espana, L., Mendez, O., Torregrosa, A., and Sierra, A. (2004). Organ-selective chemoresistance in metastasis from human breast cancer cells: inhibition of apoptosis, genetic variability and microenvironment at the metastatic focus. Carcinogenesis 25, 2293-2301.

Hanoun, M., Zhang, D., Mizoguchi, T., Pinho, S., Pierce, H., Kunisaki, Y., Lacombe, J., Armstrong, S. A., Duhrsen, U., and Frenette, P. S. (2014). Acute myelogenous leukemia-induced sympathetic neuropathy promotes malignancy in an altered hematopoietic stem cell niche. Cell stem cell 15, 365-375.

Imbimbo, B. P. (2008). Therapeutic potential of gamma-secretase inhibitors and modulators. Current topics in medicinal chemistry 8, 54-61.

Kang, Y., Siegel, P. M., Shu, W., Drobnjak, M., Kakonen, S. M., Cordon-Cardo, C., Guise, T. A., and Massague, J. (2003). A multigenic program mediating breast cancer metastasis to bone. Cancer Cell 3, 537-549.

Kellermann, S. A., and Green, L. L. (2002). Antibody discovery: the use of transgenic mice to generate human monoclonal antibodies for therapeutics. Current opinion in biotechnology 13, 593-597.

Korpal, M., Yan, J., Lu, X., Xu, S., Lerit, D. A., and Kang, Y. (2009). Imaging transforming growth factor-beta signaling dynamics and therapeutic response in breast cancer bone metastasis. Nature medicine 15, 960-966.

Kos, C. H., Karaplis, A. C., Peng, J. B., Hediger, M. A., Goltzman, D., Mohammad, K. S., Guise, T. A., and Pollak, M. R. (2003). The calcium-sensing receptor is required for normal calcium homeostasis independent of parathyroid hormone. The Journal of clinical investigation 111, 1021-1028.

Li, D., Masiero, M., Banham, A. H., and Harris, A. L. (2014). The notch ligand JAGGED1 as a target for anti-tumor therapy. Frontiers in oncology 4, 254. Lu, J., Ye, X., Fan, F., Xia, L., Bhattacharya, R., Bellister, S., Tozzi, F., Sceusi, E., Zhou, Y., Tachibana, I., et al. (2013). Endothelial cells promote the colorectal cancer stem cell phenotype through a soluble form of Jagged1. Cancer cell 23, 171-185.

Mendez, M. J., Green, L. L., Corvalan, J. R., Jia, X. C., Maynard-Currie, C. E., Yang, X. D., Gallo, M. L., Louie, D. M., Lee, D. V., Erickson, K. L., et al. (1997). Functional transplant of megabase human immunoglobulin loci recapitulates human antibody response in mice. Nature genetics 15, 146-156.

Miller, A. C., Lyons, E. L., and Herman, T. G. (2009). cis-Inhibition of Notch by endogenous Delta biases the outcome of lateral inhibition. Curr Biol 19, 1378-1383.

Nair, P., Somasundaram, K., and Krishna, S. (2003). Activated Notchl inhibits p53-induced apoptosis and sustains transformation by human papillomavirus type 16 E6 and E7 oncogenes through a PI3K-PKB/Akt-dependent pathway. Journal of virology 77, 7106-7112.

Paul, M. K., Bisht, B., Darmawan, D. O., Chiou, R., Ha, V. L., Wallace, W. D., Chon, A. T., Hegab, A. E., Grogan, T., Elashoff, D. A., et al. (2014). Dynamic changes in intracellular ROS levels regulate airway basal stem cell homeostasis through Nrf2-dependent Notch signaling. Cell stem cell 15, 199-214.

Pitt, L. A., Tikhonova, A. N., Hu, H., Trimarchi, T., King, B., Gong, Y., Sanchez-Martin, M., Tsirigos, A., Littman, D. R., Ferrando, A. A., et al. (2015). CXCL12-Producing Vascular Endothelial Niches Control Acute T Cell Leukemia Maintenance. Cancer cell 27, 755-768.

Polverino, A., Coxon, A., Starnes, C., Diaz, Z., DeMelfi, T., Wang, L., Bready, J., Estrada, J., Cattley, R., Kaufman, S., et al. (2006). AMG 706, an oral, multikinase inhibitor that selectively targets vascular endothelial growth factor, platelet-derived growth factor, and kit receptors, potently inhibits angiogenesis and induces regression in tumor xenografts. Cancer Res 66, 8715-8721.

Reedijk, M., Odorcic, S., Chang, L., Zhang, H., Miller, N., McCready, D. R., Lockwood, G., and Egan, S. E. (2005). High-level coexpression of JAG1 and NOTCH1 is observed in human breast cancer and is associated with poor overall survival. Cancer Res 65, 8530-8537.

Reedijk, M., Pinnaduwage, D., Dickson, B. C., Mulligan, A. M., Zhang, H., Bull, S. B., O→Malley, F. P., Egan, S. E., and Andrulis, I. L. (2008). JAG1 expression is associated with a basal phenotype and recurrence in lymph node-negative breast cancer. Breast Cancer Res Treat 111, 439-448.

Ren, G., Zhang, L., Zhao, X., Xu, G., Zhang, Y., Roberts, A. I., Zhao, R. C., and Shi, Y. (2008). Mesenchymal stem cell-mediated immunosuppression occurs via concerted action of chemokines and nitric oxide. Cell Stem Cell 2, 141-150.

Ridgway, J., Zhang, G., Wu, Y., Stawicki, S., Liang, W. C., Chanthery, Y., Kowalski, J., Watts, R. J., Callahan, C., Kasman, I., et al. (2006). Inhibition of Dll4 signalling inhibits tumour growth by deregulating angiogenesis. Nature 444, 1083-1087.

Rizzo, P., Osipo, C., Foreman, K., Golde, T., Osborne, B., and Miele, L. (2008). Rational targeting of Notch signaling in cancer. Oncogene 27, 5124-5131.

Roodhart, J. M., Daenen, L. G., Stigter, E. C., Prins, H. J., Gerrits, J., Houthuijzen, J. M., Gerritsen, M. G., Schipper, H. S., Backer, M. J., van Amersfoort, M., et al. (2011). Mesenchymal stem cells induce resistance to chemotherapy through the release of platinum-induced fatty acids. Cancer cell 20, 370-383.

Sethi, N., Dai, X., Winter, C. G., and Kang, Y. (2011). Tumor-derived JAGGED1 promotes osteolytic bone metastasis of breast cancer by engaging notch signaling in bone cells. Cancer Cell 19, 192-205.

Shiozawa, Y., Pedersen, E. A., Havens, A. M., Jung, Y., Mishra, A., Joseph, J., Kim, J. K., Patel, L. R., Ying, C., Ziegler, A. M., et al. (2011). Human prostate cancer metastases target the hematopoietic stem cell niche to establish footholds in mousebone marrow. The Journal of clinical investigation 121, 1298-1312.

Sprinzak, D., Lakhanpal, A., Lebon, L., Santat, L. A., Fontes, M. E., Anderson, G. A., Garcia-Ojalvo, J., and Elowitz, M. B. (2010). Cis-interactions between Notch and Delta generate mutually exclusive signalling states. Nature 465, 86-90.

Wan, L., Lu, X., Yuan, S., Wei, Y., Guo, F., Shen, M., Yuan, M., Chakrabarti, R., Hua, Y., Smith, H. A., et al. (2014). MTDH-SND1 interaction is crucial for expansion and activity of tumor-initiating cells in diverse oncogene- and carcinogen-induced mammary tumors. Cancer cell 26, 92-105.

Wang, H., Yu, C., Gao, X., Welte, T., Muscarella, A. M., Tian, L., Zhao, H., Zhao, Z., Du, S., Tao, J., et al. (2015). The Osteogenic Niche Promotes Early-Stage Bone Colonization of Disseminated Breast Cancer Cells. Cancer cell.

Wang, Z., Zhang, Y., Li, Y., Banerjee, S., Liao, J., and Sarkar, F. H. (2006). Down-regulation of Notch-1 contributes to cell growth inhibition and apoptosis in pancreatic cancer cells. Molecular cancer therapeutics 5, 483-493.

Weilbaecher, K. N., Guise, T. A., and McCauley, L. K. (2011). Cancer to bone: a fatal attraction. Nat Rev Cancer 11, 411-425.

Wu, Y., Cain-Horn, C., Choy, L., Hagenbeek, T. J., de Leon, G. P., Chen, Y., Finkle, D.,Venook, R., Wu, X., Ridgway, J., et al. (2010). Therapeutic antibody targeting of individual Notch receptors. Nature 464, 1052-1057.

Zeng, Q., Li, S., Chepeha, D. B., Giordano, T. J., Li, J., Zhang, H., Polverini, P. J., Nor, J., Kitajewski, J., and Wang, C. Y. (2005). Crosstalk between tumor and endothelial cells promotes tumor angiogenesis by MAPK activation of Notch signaling. Cancer Cell 8, 13-23.

Zheng, H., Shen, M., Zha, Y. L., Li, W., Wei, Y., Blanco, M. A., Ren, G., Zhou, T., Storz, P., Wang, H. Y., and Kang, Y. (2014). PKD1 phosphorylation-dependent degradation of SNAIL by SCF-FBXO11 regulates epithelial-mesenchymal transition and metastasis. Cancer cell 26, 358-373.

1. A method of treating cancer bone metastases comprising administering to a patient in need thereof a therapeutically effective amount of an anti-Jagged1 antibody or antigen-binding fragment thereof and a chemotherapeutic agent, wherein the anti-Jagged1 antibody or antigen-binding fragment thereof targets both tumor-derived Jagged1 and chemotherapy-induced Jagged1 in osteogenic cells.

2. The method of claim 1 wherein the cancer is breast cancer, prostate cancer, lung cancer, ovarian cancer, colorectal cancer, melanoma, multiple myeloma, thyroid cancer, bladder cancer, or kidney cancer.

3. The method of claim 2 wherein the cancer is breast cancer.

4. The method of claim 3 wherein the anti-Jagged1 antibody is a monoclonal antibody.

5. The method of claim 4 wherein the monoclonal antibody is a fully human monoclonal antibody, a humanized monoclonal antibody, or a chimeric monoclonal antibody.

6. The method of claim [[5]]4 wherein the monoclonal antibody is 15D11.

7. The method of claim 6 wherein the patient is a female human and the Jagged1 is human Jagged1.

8. The method of claim 7 wherein the chemotherapeutic agent is selected from the group consisting of alkylating agents, alkyl sulfonates, azirklines, ethylenimines, methylamelamines, colchicines, camptothecins, nitrogen mustards, nitrosoureas, plant alkaloids, bisphosphonates, anthracyclines, anti-metabolites, anti-microtubule agents, topoisomerase inhibitors, cytotoxic antibiotics, metal salts, toxoids, taxanes, pyrimidine analogs, purine analogs, aromatase inhibitors, mitomycins, androgens, anti-adrenals, folic acid replenishers, anti-folates, dihydrofolate reductase inhibitors, thymidylate synthase inhibitors, vinca alkaloids, and anti-hormonal agents, as well as pharmaceutically acceptable salts, acids, or derivatives of any of the above, as well as combinations of two or more of the above.

9. The method of claim 7 wherein the chemotherapeutic agent is paclitaxel, docetaxel, doxorubicin, epirubicin, 5-fluorouracil, cyclophosphamide, carboplatin, cisplatin, palbociclib, anastrozole, bevacizumab, capecitabine, doxorubicin liposomal injection, exemestane, gemcitabine, ixabepilone, letrozole, or trastuzumab.

10. The method of claim 7 wherein the chemotherapeutic agent is paclitaxel, 5-fluorouracil, carboplatin, or cisplatin.

11. The method of claim 7 wherein progression free survival of the patient is extended in a synergistic manner as compared to the progression free survival provided by administering the anti-Jagged1 antibody alone or the chemotherapeutic agent alone.

12. The method of claim 7 wherein the overall survival of the patient is extended in a synergistic manner as compared to the overall survival provided by administering the anti-Jagged1 antibody alone or the chemotherapeutic agent alone.

13. The method of claim 1 wherein the administration does not lead to an abnormal increase of trabecular bone density in the patient.

14. A method of providing a synergistic effect in the treatment of breast cancer comprising administering to a human female having breast cancer bone metastases a therapeutically effective amount of an anti-Jagged1 antibody or antigen-binding fragment thereof and a chemotherapeutic agent, wherein the progression free survival, the overall survival of the human female, or a combination thereof is extended beyond that provided by administering the anti-Jagged1 antibody or antigen-binding fragment thereof alone or the chemotherapeutic agent alone, wherein the anti-Jagged1 antibody or antigen-binding fragment thereof targets both tumor-derived Jagged11 and chemotherapy-induced Jagged1 in osteogenic cells.


SEQUENCE LISTING (68 sequences)

SEQ ID NO: 1 - protein, 5 aa, Artificial Sequence
  Description of Artificial Sequence Synthetic peptide
SYAMH

SEQ ID NO: 2 - protein, 17 aa, Artificial Sequence
  Description of Artificial Sequence Synthetic peptide
VISYDGSNKYYADSVKG

SEQ ID NO: 3 - protein, 17 aa, Artificial Sequence
  Description of Artificial Sequence Synthetic peptide
AIYPDSSNKYYADSVKG

SEQ ID NO: 4 - protein, 17 aa, Artificial Sequence
  Description of Artificial Sequence Synthetic peptide
AISPEASNKYYADSVKG

SEQ ID NO: 5 - protein, 17 aa, Artificial Sequence
  Description of Artificial Sequence Synthetic peptide
AIYPASSNKYYADSVKG

SEQ ID NO: 6 - protein, 11 aa, Artificial Sequence
  Description of Artificial Sequence Synthetic peptide
DKYDIPDAFDI

SEQ ID NO: 7 - protein, 11 aa, Artificial Sequence
  Description of Artificial Sequence Synthetic peptide
RASQGISNDLA

SEQ ID NO: 8 - protein, 7 aa, Artificial Sequence
  Description of Artificial Sequence Synthetic peptide
ATSTLQS

SEQ ID NO: 9 - protein, 8 aa, Artificial Sequence
  Description of Artificial Sequence Synthetic peptide
QQSYNAPI

SEQ ID NO: 10 - protein, 6 aa, Artificial Sequence
  Description of Artificial Sequence Synthetic peptide
SSNWWS

SEQ ID NO: 11 - protein, 16 aa, Artificial Sequence
  Description of Artificial Sequence Synthetic peptide
EIFHGENTNYNPSLKS

SEQ ID NO: 12 - protein, 11 aa, Artificial Sequence
  Description of Artificial Sequence Synthetic peptide
NPGIGAAKFDS

SEQ ID NO: 13 - protein, 16 aa, Artificial Sequence
  Description of Artificial Sequence Synthetic peptide
KSSQSLLHSDGKTYLY

SEQ ID NO: 14 - protein, 7 aa, Artificial Sequence
  Description of Artificial Sequence Synthetic peptide
EVSNRFS

SEQ ID NO: 15 - protein, 7 aa, Artificial Sequence
  Description of Artificial Sequence Synthetic peptide
MQHIDFP

SEQ ID NO: 16 - protein, 5 aa, Artificial Sequence
  Description of Artificial Sequence Synthetic peptide
SYWIH

SEQ ID NO: 17 - protein, 17 aa, Artificial Sequence
  Description of Artificial Sequence Synthetic peptide
RIYPGIGSTYYNEKFKD

SEQ ID NO: 18 - protein, 7 aa, Artificial Sequence
  Description of Artificial Sequence Synthetic peptide
NGGFFDY

SEQ ID NO: 19 - protein, 15 aa, Artificial Sequence
  Description of Artificial Sequence Synthetic peptide
RASESVDSYGNSFMH

SEQ ID NO: 20 - protein, 7 aa, Artificial Sequence
  Description of Artificial Sequence Synthetic peptide
RASNLES

SEQ ID NO: 21 - protein, 9 aa, Artificial Sequence
  Description of Artificial Sequence Synthetic peptide
QQSNEDPWT

SEQ ID NO: 22 - protein, 10 aa, Artificial Sequence
  Description of Artificial Sequence Synthetic peptide
GFTFSNYGIH

SEQ ID NO: 23 - protein, 16 aa, Artificial Sequence
  Description of Artificial Sequence Synthetic peptide
WITPDGGYTDYADSVK

SEQ ID NO: 24 - protein, 7 aa, Artificial Sequence
  Description of Artificial Sequence Synthetic peptide
AGSWFAY

SEQ ID NO: 25 - protein, 11 aa, Artificial Sequence
  Description of Artificial Sequence Synthetic peptide
RASQDVSTAVA

SEQ ID NO: 26 - protein, 7 aa, Artificial Sequence
  Description of Artificial Sequence Synthetic peptide
SASFLYS

SEQ ID NO: 27 - protein, 9 aa, Artificial Sequence
  Description of Artificial Sequence Synthetic peptide
QQSYTTPPT

SEQ ID NO: 28 - protein, 10 aa, Artificial Sequence
  Description of Artificial Sequence Synthetic peptide
GFTFTSYDIH

SEQ ID NO: 29 - protein, 15 aa, Artificial Sequence
  Description of Artificial Sequence Synthetic peptide
GISPADGDTDYANSV

SEQ ID NO: 30 - protein, 14 aa, Artificial Sequence
  Description of Artificial Sequence Synthetic peptide
NDYDVRSVGSGMDY

SEQ ID NO: 31 - protein, 10 aa, Artificial Sequence
  Description of Artificial Sequence Synthetic peptide
RASDVSTAVA

SEQ ID NO: 32 - protein, 7 aa, Artificial Sequence
  Description of Artificial Sequence Synthetic peptide
SASFLYS

SEQ ID NO: 33 - protein, 9 aa, Artificial Sequence
  Description of Artificial Sequence Synthetic peptide
QQSYTTPPT

SEQ ID NO: 34 - protein, 5 aa, Artificial Sequence
  Description of Artificial Sequence Synthetic peptide
DYEMH

SEQ ID NO: 35 - protein, 15 aa, Artificial Sequence
  Description of Artificial Sequence Synthetic peptide
QPGGGGTAYNQKFKG

SEQ ID NO: 36 - protein, 10 aa, Artificial Sequence
  Description of Artificial Sequence Synthetic peptide
RGYDDYPFAY

SEQ ID NO: 37 - protein, 11 aa, Artificial Sequence
  Description of Artificial Sequence Synthetic peptide
RASGNIHNYLA

SEQ ID NO: 38 - protein, 9 aa, Artificial Sequence
  Description of Artificial Sequence Synthetic peptide
NAKTLADDI

SEQ ID NO: 39 - protein, 9 aa, Artificial Sequence
  Description of Artificial Sequence Synthetic peptide
NHFWSAPWT

SEQ ID NO: 40 - protein, 5 aa, Artificial Sequence
  Description of Artificial Sequence Synthetic peptide
DYAMH

SEQ ID NO: 41 - protein, 15 aa, Artificial Sequence
  Description of Artificial Sequence Synthetic peptide
DTYYGDASYNQKFKG

SEQ ID NO: 42 - protein, 10 aa, Artificial Sequence
  Description of Artificial Sequence Synthetic peptide
LYDYDGGFAY

SEQ ID NO: 43 - protein, 16 aa, Artificial Sequence
  Description of Artificial Sequence Synthetic peptide
KSSQSLLNSSNQKNYL

SEQ ID NO: 44 - protein, 9 aa, Artificial Sequence
  Description of Artificial Sequence Synthetic peptide
FASTRRSGV

SEQ ID NO: 45 - protein, 9 aa, Artificial Sequence
  Description of Artificial Sequence Synthetic peptide
QQHYSTPYT

SEQ ID NO: 46 - protein, 5 aa, Artificial Sequence
  Description of Artificial Sequence Synthetic peptide
DYAIH

SEQ ID NO: 47 - protein, 15 aa, Artificial Sequence
  Description of Artificial Sequence Synthetic peptide
NTYYGDSKYNQKFKD

SEQ ID NO: 48 - protein, 7 aa, Artificial Sequence
  Description of Artificial Sequence Synthetic peptide
GYDGFAY

SEQ ID NO: 49 - protein, 11 aa, Artificial Sequence
  Description of Artificial Sequence Synthetic peptide
RYSENIYSYLT

SEQ ID NO: 50 - protein, 9 aa, Artificial Sequence
  Description of Artificial Sequence Synthetic peptide
NAKILAAGV

SEQ ID NO: 51 - protein, 9 aa, Artificial Sequence
  Description of Artificial Sequence Synthetic peptide
QHHYDIPWT

SEQ ID NO: 52 - protein, 5 aa, Artificial Sequence
  Description of Artificial Sequence Synthetic peptide
DYAMH

SEQ ID NO: 53 - protein, 15 aa, Artificial Sequence
  Description of Artificial Sequence Synthetic peptide
NTYYGDARYNQKFKG

SEQ ID NO: 54 - protein, 7 aa, Artificial Sequence
  Description of Artificial Sequence Synthetic peptide
GKEGFAY

SEQ ID NO: 55 - protein, 11 aa, Artificial Sequence
  Description of Artificial Sequence Synthetic peptide
RASENIYSYLA

SEQ ID NO: 56 - protein, 9 aa, Artificial Sequence
  Description of Artificial Sequence Synthetic peptide
NAKTKAEVV

SEQ ID NO: 57 - protein, 9 aa, Artificial Sequence
  Description of Artificial Sequence Synthetic peptide
QHHYGTPLT

SEQ ID NO: 58 - protein, 5 aa, Artificial Sequence
  Description of Artificial Sequence Synthetic peptide
SYAMS

SEQ ID NO: 59 - protein, 17 aa, Artificial Sequence
  Description of Artificial Sequence Synthetic peptide
SIDPEGRQTYYADSVKG

SEQ ID NO: 60 - protein, 10 aa, Artificial Sequence
  Description of Artificial Sequence Synthetic peptide
DIGGRSAFDY

SEQ ID NO: 61 - protein, 9 aa, Artificial Sequence
  Description of Artificial Sequence Synthetic peptide
RASQSISSY

SEQ ID NO: 62 - protein, 7 aa, Artificial Sequence
  Description of Artificial Sequence Synthetic peptide
AASSLQS

SEQ ID NO: 63 - protein, 9 aa, Artificial Sequence
  Description of Artificial Sequence Synthetic peptide
QQTVVAPPL

SEQ ID NO: 64 - protein, 15 aa, Artificial Sequence
  Description of Artificial Sequence Synthetic peptide
GGGGSGGGGSGGGGS

SEQ ID NO: 65 - DNA, 34 bp, Artificial Sequence
  Description of Artificial Sequence Synthetic primer
cattatttaaatgccaccatgcggtccccacgga

SEQ ID NO: 66 - DNA, 37 bp, Artificial Sequence
  Description of Artificial Sequence Synthetic primer
cattatttaaatctgctatacgatgtattccatccgg

SEQ ID NO: 67 - DNA, 21 bp, Artificial Sequence
  Description of Artificial Sequence Synthetic primer
caacaccacggaattgtcagt

SEQ ID NO: 68 - DNA, 20 bp, Artificial Sequence
  Description of Artificial Sequence Synthetic primer
gatgatgggaaccctgtcaa